(19) United States
(12) Patent Application Publication
(10) Pub. No.: US 2024/0408211 A1
(43) Pub. Date: Dec. 12, 2024

(54) COMPOSITIONS AND METHODS OF AMELIORATING AVERSIVENESS OF AVERSIVE STIMULI BY INHIBITION OF PURINERGIC RECEPTORS

(71) Applicants: Paul BRESLIN, Pittsburgh, PA (US); Monell Chemical Senses Center, Philadelphia, PA (US)

(72) Inventors: Peihua Jiang, Clarksville, MD (US); Linda Flammer, Mechanicsville, PA (US); Paul Breslin, Pittsburgh, PA (US)

(21) Appl. No.: 18/698,524

(22) PCT Filed: Oct. 4, 2022
(86) PCT No.: PCT/US2022/077554

§ 371 Date: Apr. 4, 2024

Related U.S. Application Data
(60) Provisional application No. 63/251,999, filed on Oct. 4, 2021.

(51) Int. Cl. A61K 47/22; A61K 9/00

(52) U.S. Cl. CPC A61K 47/22; A61K 9/0053

(57) ABSTRACT

Provided herein are compositions and methods of modulating an aversive taste of active pharmaceutical ingredients (API) in a medicament, nutrient, and/or a dietary supplement via inhibition of a purinergic receptor. Additionally provided, a dissolving strip, a coating applied to a food product, a chewable food product, a lollipop, and/or a liquid suspension comprising a composition comprising an effective amount of at least one compound that is a P2X3 purinergic receptor inhibitor which modulates, reduces, and/or blocks aversive taste of an active pharmaceutical ingredient in a medicament, nutrient, and/or dietary supplement that is orally administered.

### BACKGROUND OF THE INVENTION

Significant numbers of drugs and active pharmaceutical ingredients (APIs) on the market and in development may cause aversiveness upon oral administration, e.g., bitter taste, mouth/throat irritation, and nausea, not only to humans (e.g., to children and to many adults), but also to animals. Such drugs are necessary to treat global diseases such as infections with viruses, bacteria, plasmodia, and helminths, many of which are fatal. Thus, acceptable palatability of oral medicinal products is of great importance to facilitate patient adherence with a drug regimen, particularly in underdeveloped countries where medical supervision of such adherence is lacking. Unlike adults who can swallow taste-masked tablets, children are exposed to other formulations (e.g., liquid). When in liquid form, drugs can be highly aversive and children, particularly, have demonstrated little tolerance of these formulations resulting in significant problems with compliance, which may result in life-threatening consequences. For example, in cases with children who are diagnosed and treated for Human Immunodeficiency Virus (HIV), when a child does not consistently take the anti-retroviral medicines, it provides an opportunity for the virus to mutate and therefore become more difficult to treat (Schweiebert, E., et al., Molecular Pharmacology, 2021, 99(5):319-327.Holkmann, O., et al., HIV Med, 2007, 8:96-104; and Nachega, J. B., et al., Infect Disord Drug Targets, 2011, 11:167-174). If drug compliance is not improved, millions of children will continue to die from drug-treatable diseases. This has been recognized by regulatory authorities and is becoming a key aspect of pediatric pharmaceutical development studies. See, e.g., Walsh et al, “Playing hide and seek with poorly tasting pediatric medicines: Do not forget the excipients.” Advanced Drug Delivery Reviews. 73 (2014) 14-33; and Mennella et al., “Optimizing Oral Medications for Children.” Clin Ther. 2008 November, 30(11): 2120-2132.

Due to the urgent need for increasing consistent medial compliance, several approaches have been utilized to mask unpleasant tastes of APIs in pediatric oral dosage forms. However, few successes have been achieved, mainly due to high complexity of taste receptors and signal pathways involved in taste sensations, as well as little understanding thereof. In some instances, the more invasive procedures are used for pediatric drug administration, such as physically restraining and forcing a child to take the medicine, or performing percutaneous endoscopic gastrostomy to place medicine directly in the stomach of a child (Hammami, N., et al., Pediatrics, 2004, 114: e591-e597; and Shingadia, D., et al., Pediatrics, 2000, 105: E80). In other instances, flavoring and sweeteners are added to the medicine as a bitterness masking agent, but it does not always adequately mask the bitterness. Additionally, while sweeteners and flavors are the intuitive choice, such additives, when found to be effective, harm dental health or cannot be provided to diabetic children. Similarly, when salts have been previously employed to ameliorate bitterness, the degree of bitterness suppression, if any, varied widely across bitter substances, and therefore cannot be used as a universal masking agent. See, e.g., P. A. S. Breslin and G. K. Beauchamp, “Suppression of Bitterness by Sodium: Variation Among Bitter Taste Stimuli.” Chemical Senses, 1995, 20(6):609-623.

There still remains an urgent need in the art for compositions useful for modulating, reducing, and/or blocking the aversive (i.e., sweet, sour, salty, savory, and/or bitter) tasting active pharmaceutical ingredient (API), especially for use in increasing patient compliance in taking or facilitating the taking of the medicament, nutrient, and/or the dietary supplement that comprise the aversive tasting API.

### SUMMARY OF THE INVENTION

In one aspect, provided herein is a method of reducing bitter taste attributed to a bitter taste of an API in a medicament, nutrient, and/or a dietary supplement, said method comprising administration of the taste-masking composition, wherein said taste-masking composition comprising: at least one compound that is a purinergic receptor inhibitor, wherein the at least one compound modulates an aversive taste of an active pharmaceutical ingredient (API) in a medicament, nutrient, and/or a dietary supplement which is to be administered to a subject in a need thereof; and
a pharmaceutically acceptable carrier, wherein the at least one compound is in an amount sufficient to reduce and/or block the aversive taste of the API, wherein the taste-masking composition is formulated for oral administration and is administered prior to the or with the administration of the medicament and/or dietary supplement to the subject in a need thereof, and wherein said taste-masking composition is administered before the administration of the medicament, nutrient, and/or the dietary supplement to a subject in a need thereof. In certain embodiments, the method comprises administering taste-masking composition comprising at least one inhibitor targeting homomeric P2X2 receptor, homomeric P2X3 receptor, or heteromeric P2X2/P2X3 receptor. In certain embodiments, the method comprise administering taste-masking composition comprising at least one inhibitor which is a pyridine derivatives or pyrimidine derivatives, optionally wherein the inhibitor is a derivative of 5-[5-iodo-4-methoxy-2-(1-methylethyl)phenoxy]-2,4-pyrimidinediamine (AF-353), further optionally wherein the inhibitor is 5-[5-iodo-4-methoxy-2-(1-methylethyl)phenoxy]-2,4-pyrimidinediamine (AF-353).

In another aspect, provided herein is a composition which is a taste masking composition comprising at least one compound that is a purinergic receptor inhibitor, wherein the at least one compound modulates an aversive taste of an active pharmaceutical ingredient (API) in a medicament, a nutrient, and/or a dietary supplement which is to be administered to a subject in the need thereof, and a pharmaceutically acceptable carrier, wherein the at least one compound is present in an amount sufficient to reduce the aversive taste of the API, and wherein the composition is formulated for oral administration to be administered prior to or with the medicament, the nutrient, and/or the dietary supplement. In certain embodiments, the purinergic receptor inhibitor targets homomeric P2X or heteromeric P2X receptor. In certain embodiments, the purinergic receptor inhibitor targets P2X2 homomer receptor and/or P2X2/P2X3 heteromer receptor. In certain embodiments, the purinergic receptor inhibitor is selected from arylamide derivatives, diaminpyrimidine derivatives, imidazo-pyrimidine derivatives, pyrazolo-pyrimidine derivatives, pyrazole derivatives, oxazole derivatives, pyridine derivatives, pyrimidine derivatives, and physiologically or pharmaceutically acceptable salts thereof. In certain embodiments, the purinergic receptor inhibitor is 5-[5-iodo-4-methoxy-2-(1-methylethyl)phenoxy]-2,4-pyrimidinediamine (AF-353), or a derivative thereof. In some embodiments, the purinergic receptor inhibitor is present orally in an amount sufficient to reduce and/or block the aversive taste of the API. In other embodiments, the purinergic receptor inhibitor is formulated in a composition at a topical oral treatment dose (a) about 0.01 mg/mL to about 0.4 mg/mL; (b) about 0.04 mg/mL to about 0.2 mg/mL; (c) about 0.1 mg/mL to about 0.15 mg/mL.

In a further aspect, provided herein is a method of reducing bitter taste attributed to a bitter taste of an API in a medicament, nutrient, and/or a dietary supplement. In another aspect, provided herein is a method of blocking bitter taste attributed to a bitter taste of an API in a medicament, nutrient, and/or a dietary supplement. In yet another aspect, provided herein is a method of facilitating taking and/or of increasing patient compliance of taking a medicament, nutrient, and/or a dietary supplement which comprises an averse tasting API.

In yet further aspect, provided herein is a composition formulated for topical oral administration for use in facilitating taking or for use in increasing patient compliance of taking a medicament, nutrient, and/or a dietary supplement that comprises an averse tasting API. In certain embodiments, the formulation dissolving strip. In certain embodiments, the formulation is a coating applied on a food product. In certain embodiments, the formulation is a coating applied on an averse tasting API. In certain embodiments, the formulation is chewable food product. In certain embodiments, the formulation is a chewable or rapid dissolve tablet. In certain embodiments, the formulation is a liquid suspension. In certain embodiments, the formulation is an oral topical spray or an oral topical mist that coast an epithelium in oral cavity.

Other aspects and advantages of the invention will be readily apparent from the following detailed description of the invention.

### BRIEF DESCRIPTION OF THE DRAWINGS

FIG. 1 shows a graph representing perceived intensities of taste and chemesthetic stimuli before and after treatment with AF-353 oral rinse composition in humans.

FIGS. 2A to 2C show results of the trials for delivering AF-353 to the oral cavity. FIG. 2A shows influence on licking for deionized water and 1 mM quinine hydrochloride (QHCl) of the varied duration the oral cavity was swabbed (0-8 minutes). FIG. 2B shows influence on licking for deionized water and 1 mM quinine hydrochloride (QHCl) of the varied concentration of AF-353 swabbed orally for 4 min. FIG. 2C shows influence on licking for deionized water and 1 mM quinine hydrochloride (QHCl) of the interval between the end of swabbing the oral cavity with 3 mM AF-353 for 4 min and the start of gustometer testing. These results led, in subsequent experiments, to use of 4 min oral swabbing with 3 mM AF-353 then a 10 min recovery interval before the start of behavioral testing. Values are means±SEs.

FIG. 3 shows influence on licking for deionized water and 1 mM quinine hydrochloride (QHCl) of the varied concentration of AF-219 swabbed orally.

FIGS. 4A to 4B show effect of route of administration and vehicle for AF-353 on licking for deionized water and 1 mM quinine hydrochloride (QHCl). Values are means±SEs. FIG. 4A shows results of the experiment, plotted as licks per 10-sec trial, in which mice were administered 41 μmol/mouse AF-353 by oral swabbing. FIG. 4B shows results of the experiment, plotted as licks per 10-sec trial, in which mice were administered 41 μmol/mouse AF-353 by intraperitoneal injection. FIG. 4C shows results of the experiment, plotted as licks per 10-sec trial, in which mice were administered orally with AF-353 dissolved in DMSO. FIG. 4D shows results of the experiment, plotted as licks per 10-sec trial, in which mice were administered orally with AF-353 dissolved in alcohol with tween.

FIGS. 5A to 5E show results of licking for exemplars of four basic tastes and capsaicin by mice that have been orally swabbed with DMSO vehicle or 3 mM AF-353. Symbols show means±SEs; X-axis values are in mM. *p<0.05 relative to licks in vehicle-treated condition. FIG. 5A shows results of licking for sweet taste (saccharin). FIG. 5B shows results of licking for bitter taste (quinine hydrochloride (QHCL)). FIG. 5C shows results of licking for salty taste (NaCl). FIG. 5D shows results of licking for acidic (citric acid). FIG. 5E shows results of licking for capsaicin.

FIGS. 6A to 6F show licking for three bitter compounds and three medicines by mice that have been orally swabbed with DMSO vehicle or 3 mM AF-353. Symbols show means±SEs; X-axis values are in mM.*p<0.05 relative to licks in vehicle-treated condition. FIG. 6A shows results of licking for urea. FIG. 6B shows results of licking for denatonium. FIG. 6C shows results of licking for SOA. FIG. 6D shows results of licking for Tenofovir. FIG. 6E shows results of licking for Praziquantel. FIG. 6F shows results of licking for Ferroquine.

FIGS. 7A to 7E show expended various clones of P2X2/3 stable lines responding to α,β-meATP at a dose-dependent manner. FIG. 7A shows IC50 plot for P2X2/3 stable Line 2 (2.010e-006 M). FIG. 7B shows IC50 plot for P2X2/3 stable Line 6 (2.102e-006 M). FIG. 7C shows IC50 plot for P2X2/3 stable Line 9 (3.931e-006 M). FIG. 7D shows IC50 plot for P2X2/3 stable Line 12 (1.776e-006 M). FIG. 7E shows IC50 plot for P2X2/3 stable Line 24 (2.755e-006 M).

FIG. 8A shows exemplary screening assay results using one of the various clones of P2X2/3 stable lines responding to α,β-meATP in a dose-dependent manner in the presence or absence of AF-353. FIG. 8B shows another exemplary result of the screening assay for P2X2/P2X3 response to α,β-meATP in the presence or absence of AF-353 using P2X2/3 stable cell line.

### DETAILED DESCRIPTION OF THE INVENTION

Provided herein are composition and methods of modulating, reducing, and/or blocking the aversive (e.g., sweet, sour, salty, savory, and/or bitter) tasting active pharmaceutical ingredient (API) in a medicament, nutrient, and/or a dietary supplement. Also provided herein are methods and regimens for increasing the taking or facilitating adherence of the taking of a medicament, nutrient, and/or a dietary supplement that comprises an averse tasting API. Additionally provided herein are a dissolving strip, a coating applied on a food product, a coating applied on an averse tasting API, a chewable food product, a chewable or rapid dissolve tablet, a lozenge, a lollipop, a liquid suspension, and/or or an oral topical spray or an oral topical mist formulated for topical oral administration that comprises a taste-masking composition comprising at least one compound that is a purinergic receptor inhibitor that temporarily modulates, reduces, and/or blocks an aversive taste of an API or other aversive stimuli, when the stimuli is administered.

As used herein, the term “purinergic receptor”, “purinoreceptor” or any grammatical variation thereof as used herein may refer to a family of plasma membrane proteins (i.e., G protein coupled receptors (metabotropic) or ligand-gated ion channel (ionotropic)) that are involved in several cellular functions which are mediated by purine nucleotides and nucleosides (i.e., adenosine, adenosine triphosphate (ATP)). The purinergic receptor family comprises P1, P2X and P2Y receptor, of which P2X receptor is a ligand gated ion channel, the activity of which is modulated by ATP. P2X receptor family comprises P2X1, P2X2, P2X3, P2X4, P2X5, P2X6, and P2X7 receptors. Additionally, the P2X receptor may be a homomeric (P2X2, P2X2/X2 or P2X2/P2X2) or a heteromeric (P2X2/X3 or P2X2/P2X3) receptor. The heteromeric P2X receptors comprise P2X2/3, P2X4/6, and P2X1/5 receptors. P2X3 and P2X2/3 receptors are primarily found on nociceptive neurons, and have been implicated as a target for pharmaceutical development for treatment of respiratory-(i.e., cough), pain-, gastro-, and inflammation-related disorders (Ford, A. P., In pursuit of P2X3 antagonists: novel therapeutics for chronic pain and afferent sensitization, Purinergic Signaling, 2012, 8(1): S3-S26, epub Nov. 18, 2011). For example, AF-219 is a small molecule P2X3 antagonist examined for use in treatment of refractory chronic cough in non-clinical and clinical settings (Abdulgawi, R., et al., P2X3 receptor antagonist (AF-219) in refractory chronic cough: a randomized, double-blind, placebo-controlled phase 2 study, Lancet, 2015, 385: 1198-1205, ePub Nov. 25, 2014). See also, Burnstock G. Discovery of purinergic signaling, the initial resistance and Current explosion of interest. Br J Pharmacol 2012; 167: 238-55; Chen C C, Akopian A N, Sivilotti L, et al. (1995). A P2X purinoceptor expressed by a subset of sensory neurons. Nature 1995; 377: 428-31; and Marucci, G., et al., Update on novel purinergic P2X3 and P2X2/X3 receptor antagonist and their potential therapeutic applications, Expert Opinion on Therapeutic Patents, 2019, 29(12): 943-963, ePub Nov. 14, 2019.

As used herein, the term “API” is short for active pharmaceutical ingredient and refers to a chemical or biological compound which has a physiological effect when administrated in a subject in need thereof. In one embodiment, the API is an anti-malarial, anti-protozoal, anti-parasitic, anti-amoebal, anti-viral, anti-retroviral, anti-bacterial, and anti-fungal, anti-cold and flu symptoms, anti-algesia, or anti-allergy drug. In one embodiment, the API is in its levorotatory form. In another embodiment, the API is in its dextrorotatory form. In yet another embodiment, the API is a racemic mixture of levorotatory form and dextrorotatory form. In some embodiments, the API is in the form of anti-helminth medicament, herbal medicament, and essential amino-acid medicament and/or supplement.

In one embodiment, the API is a major pharmaceutical compound for treating algesia; worms, viral or bacterial infections, or, cold, flu or allergy symptoms. In one embodiment, the API is Praziquantel, Piperaquine, Dihydroartemisinin, Ritonavir, Tenofovir, Acetaminophen, Diphenhydramine, Nicotine, Caffeine, Dextromethorphan, Guaifenesin or Loratidine. In one embodiment, the API is an Over-The-Counter (OTC) pharmaceutical or a drug sharing both structural and functional similarities thereto.

As used herein, structural and functional similarities refer to two or more APIs which share at least about 80% identity of chemical groups and are able to achieve same physiological effect with a variability of less than about 10% when administered to a subject in need. Conventional methods of analyzing structure and functions of an API are known to one of skill in the art, including but not limited to mass spectrometry (MS), electron microscopy, and various pharmacokinetics and physiological analysis.

Other suitable APIs might be selected. In one embodiment, the API is an anti-algesia or analgesic compound, for example Paracetamol (acetaminophen) or Nonsteroidal anti-inflammatory drug (NSAID). Other suitable anti-algesia compounds can be found at e.g., www.drugs.com/drug-class/analgesics.html and en.wikipedia.org/wiki/Analgesic.

In one embodiment, the API is for alleviating the symptoms of cold or flu in a subject in need, comprising reduction of the frequency of cough by at least about 25%, at least about 50%, at least about 75%, or at least about 90%; relief of the muscle pain, chills, dehydration, fatigue, fever, flushing, loss of appetite, body ache, sweating, congestion, runny nose, or sneezing, by at least about 25%, at least about 50%, at least about 75%, or at least about 90%.

In one embodiment, the API is an anti-parasitic compound, for example, anti-protozoals, anti-helminthic, anti-nematodes, anti-cestodes, anti-trematodes, anti-amoebics, and anti-fungals. Other suitable anti-parasitic compound can be found at e.g., F. Matthew Kuhlmann, James M. Fleckenstein, 157—Antiparasitic Agents, Infectious Diseases (Fourth Edition), 2017, Pages 1345-1372.e2, Volume 2, Available online 12 Aug. 2016; en.wikipedia.org/wiki/Antiparasitic; and cyto.purdue.edu/cdroms/cyto2/17/chmrx/anthelmi.

In one embodiment, the API may be an antiviral for example, Abacavir, Acyclovir (Aciclovir). In some embodiments, the API is an anti-Human Immunodeficiency Virus (HIV) compound or drug. Some of the commonly used anti-HIV drugs include nucleoside/nucleotide reverse transcriptase inhibitors, NRTIs (such as emtricitabine, stavudine, ddl, ddC, d4T, 3TC, zidovudine, abacavir, tenofovir, etc); non-nucleoside reverse transcriptase inhibitors, NNRTIs (such as rilpivirine, etravirine, nevirapine, efavirenz and delavirdine); protease inhibitors, PIs (such as saquinavir, ritonavir, indinavir, nelfinavir, amprenavir, lopinavir and atazanavir); entry inhibitors, including fusion inhibitors (such as enfuvirtide, maraviroc, ibalizumab, etc) and others, such as the integrase inhibitors raltegravir, dolutegravir, and cabotegravir; attachment inhibitor fostemsavir; post-attachment inhibitor ibalizumab-uiyk; cobicistat; and combination medicines. See, e.g., hivinfo.nih.gov/understanding-hiv/fact-sheets/fda-approved-hiv-medicines.

In one embodiment, the API is an antibacterial, for example, Vancomycin, Aminoglycosides, Aminoglycosides, Ansamycins, Carbacephem, Cephalosporins, Glycopeptides, Lincosamides, Lipopeptide, Macrolides, Monobactams, Oxazolidinones, Penicillins, Penicillin combinations, Quinolones/Fluoroquinolones, Sulfonamides, Tetracyclines, Drugs against mycobacteria, and others. Other suitable antibacterial can be found in a variety of publicly available publications, such as websites, e.g., emedicinehealth.com/antibiotics/article_em, or rugs.com/article/antibiotics, en.wikipedia.org/wiki/List_of_antibiotics, or merckmanuals.com/professional/infectious-diseases/bacteria-and-antibacterial-drugs/overview-of-antibacterial-drugs, or emedexpert.com/lists/antibiotics, or emedicinehealth.com/antibiotics/article_em.htm #7_types_of_antibiotics, among others.

In one embodiment, the API is an antifungal medication, including but not limited to, Polyene antifungals, Hamycin, Imidazoles, Triazoles, Thiazoles, Allylamines, Echinocandins. Other suitable antifungal medications can be found in publicly available sources, e.g., nhs.uk/conditions/antifungal-medicines/, drugs.com/drug-class/antifungals, drugs.com/drug-class/topical-antifungals, livestrong.com/article/27116-list-antifungals/, emedexpert.com/lists/antifungals, merckmanuals.com/professional/infectious-diseases/fungi/antifungal-drugs, en.wikipedia.org/wiki/Antifungal, and/or masshealthdruglist.ehs.state. ma.us/MHDL/pubtheradetail.do?id=28.

In one embodiment, the API is an antimalarial (anti-protozoan) medication, including without limitation, e.g., quinine, Chloroquine and chloroquine phosphate, Amodiaquine and its combination with artesunate or sulfadoxine-pyrimethamine; Pyrimethamine and its combination with sulfadoxine. Other suitable anti-malarial medications can be found in publicly available sources such as mayoclinic.org/diseases-conditions/malaria/diagnosis-treatment/drc-20351190, drugs.com/condition/malaria, drugs.com/drug-class/antimalarial-combinations, en.wikipedia.org/wiki/Antimalarial_medication, cdc.gov/malaria/travelers/drugs, nap.edu/read/11017/chapter/11, medindia.net/drugs/medical-condition/malaria, canada.ca/en/public-health/services/travel-health/drugs-generic-trade-name-treatment-prevention-malaria, and/or emedicine.medscape.com/article/221134-medication.

### Compositions

Provided herein are taste masking pharmaceutical compositions, that, when provided prior to or concurrent with an API, modulate the aversive taste of said API in a medicament, nutrient, and/or dietary supplement. The pharmaceutical composition includes at least one compound that is a purinergic receptor inhibitor and a pharmaceutically acceptable carrier. In certain embodiments, the aversiveness is selected from bitterness, sourness, astringency, nausea, saltiness, savoriness (umami), and sweetness.

In certain embodiments, the taste-masking composition comprises at least one inhibitor which targets a purinergic receptor that is a homomeric purinergic receptor. In certain embodiments, the taste-masking composition comprises at least one inhibitor which targets a purinergic receptor that is a heteromeric purinergic receptor. In some embodiments, the purinergic receptor is a P2X-type purinergic receptor. In some embodiments, the taste-masking composition comprises at least one inhibitor which targets a purinergic receptor which is selected from P2X1, P2X2, P2X3, P2X4, P2X5, P2X7, P2X2/P2X3, P2X4/P2X6, P2X1/P2X5. In some embodiments, the inhibitor targets a P2X3 purinergic receptor. In other embodiments, the inhibitor targets a P2X2/3 (also referred to as P2X2/P2X3) purinergic receptor. In certain embodiments, the inhibitor targets both P2X3 and P2X2/3 receptors.

In certain embodiments, the purinergic receptor inhibitor is selected from arylamide derivatives, diaminpyrimidine derivatives, imidazo-pyrimidine derivatives, pyrazolo-pyrimidine derivatives, pyrazole derivatives, oxazole derivatives, pyridine derivatives, pyrimidine derivatives, and physiologically or pharmaceutically acceptable salts thereof. In certain embodiments, the purinergic receptor inhibitor is pyridine derivative, optionally wherein the inhibitor is a phenoxypyridine derivative. In certain embodiments, the purinergic receptor inhibitor is pyrimidine derivative, optionally wherein the inhibitor is a phenoxy-pyrimidine derivative. In some embodiments, the taste-masking composition comprises at least one inhibitor that targets a purinergic receptor, wherein the purinergic receptor inhibitor is selected from compounds including but not limited to suramin (8-[[4-methyl-3-[[3-[[3-[[2-methyl-5-[(4,6,8-trisulfonaphthalen-1-yl) carbamoyl]phenyl]carbamoyl]phenyl]carbamoylamino]benzoyl]amino]benzoyl]amino]nap hthalene-1,3,5-trisulfonic acid); PPADS (pyridoxalphosphate-6-azophenyl-2′,4′-disulfonic acid tetrasodium salt); 2′,3′-O-(2,4,6-trinitrophenyl)adenosine-5′-triphosphate; A-317491 (5-[[[(3-phenoxyphenyl)methyl][(1S)-1,2,3,4-tetrahydro-1-naphthalenyl]amino]carbonyl]-1,2,4-benzenetricarboxylic acid); RO3 (5-(2-isopropyl-4,5-dimethoxybenzyl)pyrimidine-2,4-diamine); RO4 (5-(5-iodo-2-isopropyl-4-methoxyphenoxy)pyrimidine-2,4-diamine); MK-3901 (N-[1(R)-(5-fluoropyridin-2-yl)ethyl]-3-(5-methylpyridin-2-yl)-5-[5(S)-(2-pyridyl)-4,5-dihydroisoxazol-3-yl]benzamide); gefapixant (5-[(2,4-diaminopyrimidin-5-yl)oxy]-2-methoxy-4-(propan-2-yl)benzene-1-sulfonamide) (AF-219 or MK-7264) or (5-(2,4-diaminopyrimidin-5-yloxy)-4-isopropyl-2-methoxy-benzenesulfonamide); AF-353 (5-[5-iodo-4-methoxy-2-(1-methylethyl)phenoxy]-2,4-pyrimidinediamine); 3-(2,5-dimethoxyphenethyl)-N,N-dimethyl-4-oxo-3,4,5,6,7,8-hexahydrobenzo[4,5]thieno[2,3-d]pyrimidine-7-carboxamide; AZ-2 (N—((R)-1-(6-fluoropyridin-3-yl)ethyl)-3-(5-methylpyridin-2-yl)-5-(5-(pyridin-3-yl)-4,5-dihydroisoxazol-3-yl)benzamide); N-(4-(3-(2,5-dimethoxyphenethyl)-4-oxo-3,4-dihydroquinazolin-6-yl)-3-methoxyphenyl)acetamide; 4-oxo-quinazoline; 4-(2,4-diaminopyrimidin-5-yloxy)-2-iodo-5-isopropyl-phenol; 3-hydroxy-5-methyl-6-(3-phenoxybenzyl)-2-propylisonicotinic acid; BLU-5937 ((methyl (S)-3-((2-(2,6-difluoro-4-(methylcarbamoyl)phenyl)-7-methylimidazo [1,2-a]pyridin-3-yl)methyl)piperidine-1-carboxylate); Bellus Health, Inc.); N-00588; A-317491 (Abbott); AF-010 ((5-(2-isopropyl-4,5-dimethoxy-phenoxy)-pyrimidine-2,4-diamin); DT-0111 (3′-deoxy-3′-(3,5-dimethoxybenzamido), 5-cyclohexyl-3-hydroxy-1-(4-(isoxazol-4-yl)phenyl)-4-(4-methoxybenzoyl)-1,5-dihydro-2H-pyrrol-2-one (Shionogi), AF-001, AF-130, AF-101, AF-906, AF-454, AF-014 (Afferent Pharmaceuticals); GSK1482160 ((S)—N-(2-chloro-3-(trifluoromethyl)benzyl)-N-methyl-5-oxopyrrolidine-2-carboxamide); opiranserin (VVZ149; 4-butoxy-N-[[4-(dimethylamino)oxan-4-yl]methyl]-3,5-dimethoxybenzamide;hydrochloride); 1,3-thiazol-2-yl- substituted benzamide compounds; and derivatives thereof. See also, Marucci, G., et al., Update on novel purinergic P2X3 and P2X2/X3 receptor antagonist and their potential therapeutic applications, Expert Opinion on Therapeutic Patents, 2019, 29(12): 943-963, ePub Nov. 14, 2019; and U.S. Pat. No. 10,183,937 B2 which are incorporated herein by reference in its entirety. Inhibitors for purinergic receptors, i.e., P2X3, P2X2/3, P2X7, have been previously described. See, e.g., U.S. Pat. No. 7,858,632 B2; U.S. Pat. No. 8,008,313 B2; U.S. Pat. No. 8,846,705 B2; U.S. Pat. No. 8,946,439 B2; U.S. Pat. No. 9,896,439 B2; U.S. Pat. No. 9,556,127 B2 (Roche); U.S. Pat. No. 10,195,198B2; U.S. Pat. No. 10,662,162 B2; U.S. Pat. No. 10,676,444 B2; U.S. Pat. No. 10,822,311 B2; U.S. Pat. No. 10,988,460 B2; WO2017/058645 A1 (Afferent Pharmaceuticals); U.S. Pat. No. 6,831,193 B2; U.S. Pat. No. 7,704,997 B1; U.S. Pat. No. 7,723,367 B2; U.S. Pat. No. 8,546,374 B2; U.S. Pat. No. 8,436,005 B2; U.S. Pat. No. 8,217,067 B2 (Abbott Laboratories); U.S. Pat. No. 8,946,439 B2 (Evotec OAI AG); U.S. Pat. No. 8,598,209 B2 (Merck); U.S. Pat. No. 8,946,231 B2 (Merck); U.S. Pat. No. 9,464,084 B2 (Janssen Pharmaceuticals).

In certain embodiments, the taste-masking composition comprising a derivative of AF-353 (5-[5-iodo-4-methoxy-2-(1-methylethyl)phenoxy]-2,4-pyrimidinediamine). See also, U.S. Pat. No. 9,556,127 B2 (i), U.S. Pat. No. 10,822,311 (ii), U.S. Pat. No. 10,195,198B2 (iii) (which were also incorporated above) and WO 2007/025925 A1, which are incorporated herein by reference in their entireties, including formula (i), (ii), or (iii):

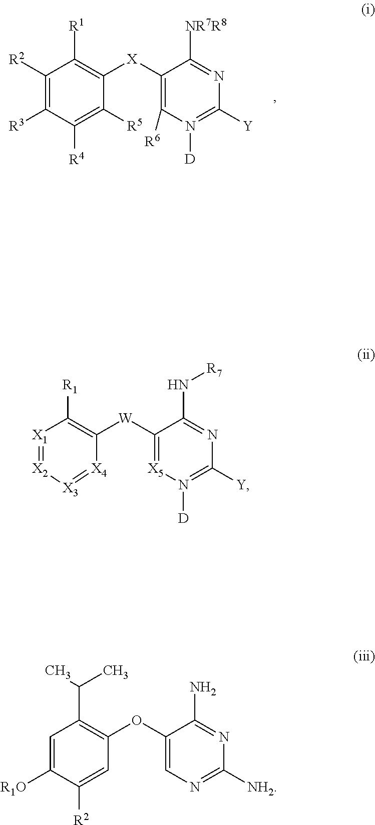

In certain embodiments, the taste-masking composition comprises at least one inhibitor that targets a homomeric P2X2, homomeric P2X3, or a heteromeric P2X2/P2X3 receptor. In some embodiments, the taste-masking composition comprises at least one inhibitor that targets a P2X2/P2X3 receptor. In further embodiments, the taste-masking composition comprises a phenoxy-pyrimidine derivative of a 5-[5-iodo-4-methoxy-2-(1-methylethyl)phenoxy]-2,4-pyrimidinediamine, including formula (i), (ii), (iii), wherein X, W, Y, D, R are including but not limited to:
X is: —CH₂—; —O—; —S(O)ₙ—; or —NR^{c}— wherein n is from 0 to 2 and Rc is hydrogen or alkyl;
W is: O or S;
Y is: hydrogen; or —NR^{d}R^{e} wherein one of R^{d} and R^{e} is hydrogen, and the other is: hydrogen; alkyl; cycloalkyl; cycloalkylalkyl; haloalkyl; haloalkoxy; hydroxyalkyl; alkoxyalkyl; acetyl; alkylsulfonyl; alkylsulfonylalkyl; aminocarbonyloxyalkyl; hydroxycarbonylalkyl; hydroxyalkyloxycarbonylalkyl; aryl; aralkyl; arylsulfonyl; heteroaryl; heteroarylalkyl; heteroarylsulfonyl; heterocyclyl; or heterocyclylalkyl;
D is an optional oxygen; and
R is: hydrogen; alkyl; alkynyl; alkenyl; amino; halo; amido; haloalkyl; alkoxy; hydroxy; haloalkoxy; nitro; isopropyl; hydroxyalkyl; hydroxyalkoxy; hydroxycarbonylalkyl; hydroxyalkyloxycarbonylalkyl; alkoxyalkyl; alkynylalkoxy; alkylsulfonyl; alkylsulfonylalkyl; alkylcarbonyl; arylsulfonyl; cyano; aryl; heteroaryl; heterocyclyl; heterocyclylalkoxy; heteroaryloxy; heteroaralkyloxy; heteroarylalkyl; heteroarylsulfonyl; aryloxy; aralkyl; arylsulfonyl; aralkyloxy; phenoxy; cycloalkenyl; cycloalkyl; cycloalkylalkyl; acetyl; aminocarbonyloxyalkyl; aminosulfonyl; or carboxyalkyl. See also, U.S. Pat. Nos. 9,556,127 B2, 10,822,311, and 10,195,198B2.

In some embodiments, the taste-masking composition comprises at least one inhibitor that targets a P2X2/P2X3 receptor, the taste-masking composition comprising a derivative of 5-[(2,4-diaminopyrimidin-5-yl)oxy]-2-methoxy-4-(propan-2-yl)benzene-1-sulfonamide (AF-219).

In some embodiments, the taste-masking composition comprises at least one inhibitor that targets a P2X2/P2X3 receptor, the taste-masking composition comprising a derivative of Methyl (S)-3-((2-(2,6-difluoro-4-(methylcarbamoyl)phenyl)-7-methylimidazo[1,2-a]pyridin-3-yl)methyl)piperidine-1-carboxylate (BLU-5937). In some embodiments, the taste-masking composition comprises at least one inhibitor that targets a P2X2/P2X3 receptor, the taste-masking composition comprising a derivative of N-00588. See also, WO 2014/117274, WO 2020/174283A1, U.S. Pat. No. 10,111,883 B1, and Garceau, D., et al., BLU-5937: A selective P2X3 antagonist with potent anti-tussive effect and no taste alteration, 2019, Pulmonary Pharmacology and Therapeutics 56 (2019): 56-62, which are incorporated herein by reference in their entireties.

In certain embodiments, the taste-masking composition comprises an AF-353 (5-[5-iodo-4-methoxy-2-(1-methylethyl)phenoxy]-2,4-pyrimidinediamine) and a pharmaceutically acceptable carrier, wherein the AF-353 is present in an amount sufficient to modulate, reduce, and/or block the averse taste of the API.

In certain embodiments, the taste-masking composition is formulated to comprise a purinergic receptor inhibitor at a dose (i.e., dosage unit or concentration) of about 0.01 mg/mL to about 0.4 mg/mL. In certain embodiments, the taste-masking composition is formulated to comprise a purinergic receptor inhibitor at a dose of about 0.04 mg/mL to about 0.2 mg/mL. In certain embodiments, the taste-masking composition is formulated to comprise a purinergic receptor inhibitor at a dose of about 0.1 mg/mL to about 0.15 mg/mL.

In certain embodiments, the taste-masking composition is formulated to comprise a purinergic receptor inhibitor at a dose (i.e., dosage unit or concentration) as measured in molar concentration (i.e., mol/L). In certain embodiments, the taste-masking composition is formulated to comprise a purinergic receptor inhibitor at a topical treatment dose (i.e., dosage unit or concentration) about 0.000001 M, about 0.000005 M, about 0.00001 M, about 0.00002 M, about 0.00003 M, about 0.00004 M, about 0.00005 M, about 0.00006 M, about 0.00007 M, about 0.00008 M, about 0.00009 M, about 0.0001 M, about 0.00015 M, about 0.0002 M, about 0.00025 M, about 0.0003 M, 0.00035 M, about 0.0004 M, about 0.00045 M, about 0.0005 M, about 0.00055 M, about 0.0006 M, about 0.00065 M, about 0.0007 M.

In certain embodiments, the taste-masking composition is formulated for oral administration. In certain embodiments, the taste masking composition is formulated in a liquid, a solid, or semi-solid form. In some embodiments, the liquid is selected from the group consisting of emulsions, microemulsions, solutions, suspensions, syrups (e.g., syrup concentrates), linctuses, drops, sprays, and elixirs. In certain embodiments, the taste-masking composition is formulated in a liquid form for use in a regimen comprising at least one rinse and expectoration. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations.

In certain embodiments, the taste-masking composition is formulated for topical oral administration in the form of dissolving strip, a coating applied on a food product, coating applied on an averse tasting API, a chewable food product, a chewable or rapid dissolve tablet, a lollipop (or lozenges), or a liquid suspension (e.g., mouthwash, oral/buccal topical spray, or mist).

The composition may be provided in any suitable formulation which delivers the taste masking composition and, optionally, the API, to the oral cavity. In one embodiment described herein, the pharmaceutical composition slowly dissolves in a subject's oral cavity and releases the taste masking composition over a prolonged period. In another embodiment described herein, the pharmaceutical composition quickly dissolves in a subject's oral cavity and releases the active pharmaceutical ingredient over a prolonged period. In one aspect, the pharmaceutical composition can be chewed or masticated. In another aspect, the pharmaceutical composition can be sucked. In another aspect, the pharmaceutical composition can be allowed to slowly dissolve in the oral cavity. In another aspect, the pharmaceutical composition can be masticated, sucked, and/or allowed to passively dissolve in the oral cavity at the subject's discretion. In another aspect, the pharmaceutical composition can be swished around in the oral cavity and either swallowed or expectorated.

As used herein, the terms physiologically or pharmaceutically acceptable carrier include any and all solvents, dispersion media, coatings, antibacterial and antifungal agents, isotonic and absorption delaying agents, adjuvants, surfactants, and the like, compatible with administration to humans, especially those suitable for oral administration. Carriers include those identified in texts such as Remington's Pharmaceutical Sciences, 17th edit., 1985 Gennaro, A R eds., Mack Publishing Co, Easton PA; and Handbook of Pharmaceutical Excipients, 6th edit., 2009 Rowe RC et al, eds, Pharmaceutical Press, incorporated by reference herein. In certain embodiments, a suitable pharmaceutical acceptable carrier may be readily selected by one of skill in the art in view of the purinergic receptor inhibitor used, and may include without limitation, a diluent, an excipient, a vector, a stabilizer, a buffer, a preservative, a sweetener, a flavor, a taste receptor antagonist, a taste transduction cascade blocker, and/or an adjuvant. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In one embodiment, provided herein composition which is a taste masking composition comprising an effective amount of at least one compound that is a purinergic receptor inhibitor, wherein the effective amount is sufficient to modulate the aversive tasting API. In certain embodiments, the effective amount of the purinergic receptor inhibitor is sufficient to reduce the averse tasting API. In certain embodiments, the effective amount of the purinergic receptor inhibitor is sufficient to block the averse tasting API.

In certain embodiments, the purinergic receptor inhibitor in combination with the medicament comprising an aversive tasting API is in a variety of orally administered formulations. The orally administered formulations may be swallowed immediately, slowly dissolved in the mouth, or chewed. Non-limiting examples of formulations can include a liquid medication, particles suspended in a liquid formulation, a solid in a gelatin or foam, or a solid dose in the form of a tablet, powder, granules, pellets, microspheres, nanospheres, beads, or nonpareils, and combinations thereof. See also, U.S. Pat. No. 9,827,320 B2. In certain embodiments, the purinergic receptor inhibitor in combination with the medicament comprising an aversive tasting API is in a liquid, solid or semi-solid formulation. In certain embodiments, the purinergic receptor inhibitor in combination with the medicament is provided as a lozenge. Soft lozenges comprising APIs are known in the art. For example, the medicament can be an oral pharmaceutical composition suitable for chewing, sucking, or buccal dissolution, e.g., a soft lozenge. See e.g., U.S. Pat. No. 9,877,971 B2 “Soft lozenges comprising corticosteroids”. Such soft lozenges provide topical, non-systemic delivery of corticosteroids to the esophagus and oral cavity. A soft lozenge comprising the purinergic receptor inhibitor optionally in combination with the API is provided in one embodiment.

In some embodiments, the taste-masking composition is co-administered to subject in the need thereof at the same time as administration of a medicament, nutrient, and/or a dietary supplement which causes aversiveness (i.e., comprising an aversive tasting API) in a subject upon oral administration thereof. In certain embodiments, the composition comprises a medicament, nutrient, and/or a dietary supplement which comprises an API that causes aversiveness in a subject upon oral administration thereof, and an effective amount of a purinergic receptor inhibitor, wherein the composition suppresses aversiveness of the API. In certain embodiments, the suppression of aversiveness includes modulation, reduction, or blocking. The aversiveness that is suppressed comprises perceived bitterness, sourness, astringency, saltiness, sweetness, savoriness (umami) and nausea. In certain embodiments, the composition comprises the taste-masking compositional and a medicament in an oral soft lozenge, wherein the oral soft lozenge comprises a shell encapsulating a semi solid matrix fill, wherein the shell comprises the taste-masking composition including (a) one or more first film-forming polymers; (b) one or more first plasticizers; (c) one or more first pH modifiers; (d) at least one compound that is a purinergic receptor inhibitor; and (e) one or more first solvents; and the matrix fill comprising: (f) one or more second film-forming polymers; (g) one or more release modifiers; (h) one or more second plasticizers; (i) one or more second pH modifiers; (j) one or more second sweeteners; (k) one or more second solvents; and (1) one or more active pharmaceutical ingredients. In some embodiments, the shell further comprises: (m) one or more opacifiers, coloring agents, flavorings, or combinations thereof; and the matrix comprises: (n) one or more solubilizing agents; and (o) one or more second active pharmaceutical ingredients. In certain embodiments, the shell or the matrix further comprises one or more pharmaceutically acceptable excipients. In another aspect, the film-forming polymer comprises one or more of gelatin, partially hydrolyzed gelatin, hydrolyzed gelatin, hydrolyzed collagen, or combinations thereof.

In certain embodiments, the composition comprises a medicament, nutrient, and/or a dietary supplement comprising an aversive tasting API, wherein medicament is suitable for oral administration, and wherein the composition further comprises a rapid dissolve coating comprising the taste-masking composition. See e.g., WO 2019/246256, which is incorporated herein by reference. In certain embodiments, the coating comprising the taste-masking composition is applied to a food product. In certain embodiments., the coating comprising the taste-masking composition is applied on an averse tasting API. In some embodiments, the taste-masking composition is administered to subject in the need thereof prior to the administration of a medicament, nutrient, and/or a dietary supplement which causes aversiveness (i.e., comprising an aversive tasting API) in a subject upon oral administration thereof. In one embodiment, the taste-masking composition is in a liquid formulation to be administered prior to the administration of a medicament, nutrient, and or the dietary supplement. In another embodiment, the taste masking composition is in a solid formulation (dissolving strip or lollipop) to be administered prior to the administration of a medicament, nutrient, and or the dietary supplement. In certain embodiments, the taste masking composition is formulated in a solid or semi-solid edible medium that dissolves upon oral contact. The formulation may further comprise a flavor agent, an antiseptic or an anesthetic. The medium may be water, a gel or pudding, or a mixture thereof. See also, US2004/0265359A1.

In certain embodiments, the taste-masking composition is formulated in a chewable or rapid dissolve (also referred to a rapid dissolution) tablet. In certain embodiments, the chewable or rapid dissolve tablet is a quick release pharmaceutical composition for oral administration comprising a taste-masking composition comprising at least one compound that is a purinergic receptor inhibitor wherein the at least one purinergic receptor inhibitor is released at a rate of at least about 50% w/w of the active substance being released within the first 20 min of application and/or administration. In certain embodiments, the chewable or rapid dissolve tablet undergoes rapid disintegration in the oral cavity allowing for enhanced coating of the oral cavity epithelium. In certain embodiments, the chewable or rapid dissolve tablet is a melt-combining a mixture of a taste-masking composition and a sugar or a sugar alcohol having numerous inherent pores through filling the mixture in a packaging material and heating to form a rapidly disintegrating formulation possessing inherent pores, alternatively, a conventional method for creating pores through sublimation, evaporation or dehumidification may be used. See also, WO 2009/002084A2 and WO 2000/015195A1, which are incorporated herein by reference in its entirety.

In certain embodiments, the taste-masking composition is formulated in a liquid suspension which is a neutral tasting oral rinse liquid suspension. In certain embodiments, the taste masking composition as described herein is in the form of a neutral tasting mouthwash. In some embodiments, the liquid suspension comprising the taste-masking composition comprises an acceptable carrier for oral use. In some embodiments, the liquid suspension comprising the taste-masking composition further comprises solvents, wherein solvents are selected from water, ethanol, glycerol, propylene glycol, polyethylene glycol 400, polyethylene glycol 200, and mixtures thereof. In certain embodiments, the liquid suspension comprises from about 40% to about 95% solvent, in another example from about 50% to about 80% solvent, and in another example from about 55% to about 60% solvent, and in another example from about 68% solvent to about 72% solvent. In certain embodiments, the liquid suspension is an oral liquid reparation such as spray or rinse. In certain embodiments, the liquid suspension is a neutral tasting mouthwash is a water-alcohol mixture comprising the taste-masking composition as describe herein, and optionally a vehicle (i.e., the carrier for the ingredients of the mouthwash, such as the essential oils, and the like). In some embodiments, the ratio of water to alcohol is in the range of from about 1:1 to about 20:1, preferably about 3:1 to about 10:1 by weight. The total amount of water-alcohol mixture in a mouthwash preparation is typically in the range from about 50% to about 99.9% by weight of the composition. The pH value of such mouthwash preparations is generally from about 3.5 to about 8.0 and preferably from about 4 to about 7.5. A pH below 3.5 would be irritating to the oral cavity and soften tooth enamel. A pH greater than 8 would result in an unpleasant mouth feel. In certain embodiments, the mouthwash further comprises surfactants, wherein surfactants are in amounts up to about 5%. Surfactants are organic materials which aid in the complete dispersion of the preparation throughout the oral cavity. The organic surfactant material may be anionic, non-ionic, ampholytic, or cationic and may be selected from among those well known in the art. See also, U.S. Pat. No. 9,827,320 B2 (Compositions with reduced bitter taste perception), U.S. Pat. No. 6,306,372B1 (Oral hygiene compositions which mask the burn sensation and the astringency of eucalyptol and zinc) which are incorporated herein by reference in its entirety.

In certain embodiments, the taste-masking composition further comprises a coloring agent, a dissolving agent, a flavoring agent, a sweetener, a viscosity modifier, an electrolyte, a vitamin, a mineral, an antioxidant, or a preservative. In certain embodiments, the taste-masking composition further comprises a formulation base. In some embodiments, the formulation base comprises a lipophilic additive. In some embodiments, the formulation base comprises an oil and a lipophilic additive. In certain embodiments, the oil is selected from vegetable oil, mineral oil, soya oil, sunflower oil, corn oil, olive oil, nut oil, and liquid paraffin. In certain embodiments, the lipophilic additive is selected from polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax.

In certain embodiments, the taste-masking composition is formulated in a liquid suspension which is an oral (buccal) topical spray (i.e., mouth spray) or an oral (buccal) topical mist. In certain embodiment, the oral topical spray or an oral topical mist coats the epithelium in the oral cavity. In certain embodiments, the oral topical spray (i.e., mouth spray) or the oral topical mist is a buccal aerosol spray comprising a polar or non-polar solvent for rapid absorption through the oral epithelium, resulting in fast onset of effect. In certain embodiments, the buccal aerosol spray compositions of the present invention further comprise in weight % of total composition: pharmaceutically acceptable propellant 5-80%, nonpolar solvent 19-85%, active compound 0.05-50% (i.e., taste masking composition comprising at least one compound that is a purinergic receptor inhibitor), suitably additionally comprising, by weight of total composition a flavoring agent 0.01-10%. Preferably the composition comprises: propellant 10-70%, non-polar solvent 25-89.9%, active compound 0.01-40%, flavoring agent 1-8%; most suitably propellant 20-70%, non-polar solvent 25-74.75%, active compound 0.25-35%, flavoring agent 2-7.5%. See also, WO 2005/032519A1, and WO 2005/032520A1, which are incorporated herein by reference. In certain embodiments, the oral topical spray (i.e., mouth spray) or the oral topical mist components typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). In certain embodiments, the oral topical spray (i.e., mouth spray) or the oral topical mist can be in a form that is directly deliverable or applicable to oral cavity. These compositions and/or preparations can be delivered by a delivery device selected from droppers, pump, sprayers, liquid dropper, cup, bottle, pressurized sprayers, atomizers, and other packaging and equipment, and combinations thereof. The sprayer, and/or atomizer can be associated with a battery or electric power source. For example, the respiratory compositions can be used to provide instant or on demand relief of taste-aversion to a human when needed. See also, WO 2008/126057A2, which is incorporated herein by reference.

In some embodiments, the taste-masking composition is in the form of a dissolving strip, wherein the taste-masking composition comprises an effective amount of a purinergic receptor inhibitor sufficient to block aversive taste of the API, and a pharmaceutical acceptable carrier including a dissolving agent, a food-grade coloring agent (e.g., purple), and a flavoring agent (e.g., grape). See also, U.S. Pat. No. 10,307,397 B2 (Oral dissolvable film that includes plant extract), U.S. Pat. No. 10,632,164 B2 (Ingestible films having substances from hemp or cannabis) which are incorporated herein by reference in its entirety. In certain embodiment, the taste masking composition further comprises a sweetening agent, which is a sugar or a sugar-alternative that is compatible with administration in diabetic subjects. In certain embodiments, the effective amount of the purinergic receptor inhibitor is varied based in the aversiveness of an API to be administered. In certain embodiments, the effective amount of the purinergic receptor inhibitor is varied based on the timing of the administration of the taste masking composition and the administration of a medicament, nutrient, and/or a dietary supplement comprising the aversive tasting API.

In certain embodiments, the dissolving strip comprising the taste-masking composition is formulated as the orally dissolving film for use prior to the prior to the administration of a medicament, nutrient, and/or a dietary supplement, wherein the orally dissolving film comprises (a) solvent that includes water, (b) binder that includes pectin, (c) optionally a lipid that includes at least one of deodorized cocoa butter oil, fruit seed oil, and vegetable based oil, (d) emulsifier that includes at least one of glycerin, ethoxylated monoglycerides, and ethoxylated diglycerides, (e) taste-masking composition comprising the at least one compound that is a purinergic receptor inhibitor, (f) flavoring agent that includes mint flavoring, (g) sweetener that includes at least one of sucralose and acesulfame potassium, (h) dye or pigment that includes at least one of FD&C red. FD&C blue, and FD&C yellow, (i) a powder coating on at least one external surface, that includes at least one of talc, microcrystalline cellulose, mint flavoring, sucralose, acesulfame potassium, and tapioca starch, and j) optionally a preservative that includes at least one of sodium benzoate, methyl paraben, propyl paraben, and sodium sorbate. The at least one compound that is a purinergic receptor in the taste-masking composition can optionally be at least partially encapsulated by the lipid. In some embodiments, the solvents are selected from water, ethanol, glycerol, propylene glycol, polyethylene glycol 400, polyethylene glycol 200, and mixtures thereof.

In certain embodiments, the dissolving strip is a thin mucosally dissolvable film comprising an emulsion or a matrix within which the taste-masking composition comprising the least one compound that is a purinergic receptor inhibitor is dispersed. In certain embodiments, the matrix comprising the at least one compound that is a purinergic receptor inhibitor may be formed from an edible polymer that is natural such as, but not limited to, methylcellulose, hydroxypropylmethylcellulose, ethylcellulose, sodium alginate, starch, chitosan, chitin, pullalan, agar, derivatives and/or combinations thereof. The matrix may also be formed from synthetic polymers including, but not limited to, hydroxyethylcellulose, cellulose acetate phthalate, hydroxypropyl methylcellulose phthalate, carboxymethyl ethylcellulose, hydroxypropylmethyl cellulose acetate succinate, polyvinyl acetate phthalate, maltodextrin, dextran, hydroxypropyl cellulose, sodium carboxymethyl cellulose, poly(methacrylic acid-co-ethyl acrylate), poly(methacrylic acid-co-methyl methacrylate), poly(methacrylic acid-co-ethyl acrylate), poly(methacrylic acid-co-methyl methacrylate), polyvinylpyrrolidone, polylactic acid (PLA), poly-L-lactide (PLLA), poly-D-lactide (PLDA), poly(lactic-co-glycolic acid) (PLGA), and mixtures thereof. In certain embodiments, the dissolvable film may additionally include permeability enhancers in amounts from about 0.001% to about 10% by weight of the film and may be selected from the group of one or more calcium chelators, polycarboxylic acids, zonula occluding toxin, poly-L-arginine, chitosan derivatives, niacin, omega 3 or 6 fatty acids or other fatty acids, menthol, sodium caprate, sodium deoxycholate, dipotassium glycyrrhizinate, 25 furanocoumarins and grapefruit derivatives, bile salts, ethylenediaminetetraacetic acid (EDTA), tocopheryl polyethyleneglycol succinate (TPGS), derivatives thereof, and combinations thereof, or the like.

In some embodiments, the taste-masking composition is in the form of a lollipop or popsicle, wherein the taste-masking composition comprises an effective amount of a purinergic receptor inhibitor sufficient to block aversive taste of the API, and a pharmaceutical acceptable carrier including a dissolving agent, a food-grade coloring agent (e.g., purple), and a flavoring agent (e.g., grape). In certain embodiment, the taste masking composition further comprises a sweetening agent, which is a sugar or a sugar-alternative that is compatible with administration in diabetic subjects. In certain embodiments, the effective amount of the purinergic receptor inhibitor is varied based in the aversiveness of an API to be administered. In certain embodiments, the effective amount of the purinergic receptor inhibitor is varied based on the timing of the administration of the taste masking composition and the administration of a medicament, nutrient, and/or a dietary supplement comprising the aversive tasting API. Lollipop formulations are generally described in U.S. Pat. Nos. 4,671,953; 4,863,737; 6,165,495 which are incorporated by reference in its entirety. In certain embodiment, the lollipop comprises a holder and a confectionary matrix attached to one end of the holder, wherein the confectionary matrix is a soluble matrix material into which taste-masking composition comprising the at least one compound that is a purinergic receptor inhibitor is dispersed throughout, wherein the confectionary matrix being capable of releasing the at least one compound that is a purinergic inhibitor for absorption in oral cavity.

In certain embodiments, the dissolving strip comprising the taste-masking composition is formulated for topical oral administration for use in facilitating taking a medicament, nutrient, and/or a dietary supplement that comprises an averse tasting API to a subject in a need thereof. In certain embodiments, the dissolving strip comprising the taste-masking composition is formulated for topical oral administration for use in increasing compliance in taking a medicament, nutrient, and/or a dietary supplement that comprises an averse tasting API to a subject in a need thereof. In certain embodiments, the subject in the need thereof is a child or an elderly.

In some embodiments, the taste-masking composition, as described herein, is for use in facilitating taking a medicament, nutrient, and/or a dietary supplement that comprises an averse tasting API to a subject in a need thereof. In certain embodiments, the subject in need thereof is a child. In certain embodiments, the subject in need thereof is an elderly. In certain embodiments, the subject in need thereof is an animal (e.g., livestock, including poultry, cattle, horse, sheep, swine and goats, rabbit, other animals in agriculture, and other animals such as fish, shrimp, and other animals in aquaculture). In certain embodiments, the subject in the is a non-human domesticated animals (i.e., canine, feline).

In certain embodiment, the taste-masking composition, as described herein, is for use to be administered to any mucosal surface of the animal, preferably a membrane of the oral mucosa, including lingual surfaces, sublingual surfaces, buccal surfaces, palatal surfaces, and pharyngeal surfaces, preferably buccal or gingival surfaces. In yet other embodiments, the compositions of the present invention can be administered to the area of the oral cavity of an animal between the teeth and cheek.

It should be understood that any embodiment of the composition provided herein may be utilized in any other embodiment, composition, method or kit described herein.

### Methods and Kits

In one aspect, a method is provided herein of modulating, inhibiting, reducing or blocking an aversiveness in the oral cavity caused by exposure to or ingestion of medicament, nutrient, and/or a dietary supplement that induces the aversiveness, comprising involves contacting the oral cavity with taste-masking composition comprising an effective amount of at least one compound that is a purinergic receptor inhibitor, before exposure or after exposure to the indicated medicament, nutrient, and/or a dietary supplement. In another aspect, provided herein is a method of facilitating taking a medicament, nutrient, and/or a dietary supplement which comprises an averse tasting API. In yet another aspect, provided herein is a method of increasing patient compliance of taking a medicament, nutrient, and/or a dietary supplement which comprises an averse tasting API.

In certain embodiment, the method comprises administering the medicament, nutrient, and/or a dietary supplement and the taste masking composition as described herein comprising an effective amount of purinergic receptor inhibitor orally to the subject. In one embodiment, the taste-masking composition comprising a purinergic receptor inhibitor is added to the formulation of the indicated medicament, nutrient, and/or a dietary supplement upon administration. In another embodiment, the taste-masking composition is administered to the subject prior to the administration of the drug. In certain embodiments, the taste-masking composition is administered at least about 1 hour, at least about 30 minutes, at least about 15 minutes, at least about 10 minutes, or at least about 5 minutes prior to the administration of the medicament, nutrient, and/or the dietary supplement. In certain embodiments, the taste-masking composition is administered at least 30 minutes prior to administration of a medicament, nutrient, and/or a dietary supplement to a subject in the need thereof. In certain embodiments, the taste-masking composition is administered at least 15 minutes prior to administration of a medicament, nutrient, and/or a dietary supplement to a subject in the need thereof. In certain embodiments, the taste-masking composition is administered at least 10 minutes prior to administration of a medicament, nutrient, and/or a dietary supplement to a subject in the need thereof. In certain embodiment, the subject is a child, an adult, or an elderly person. In certain embodiment, the subject is an animal, optionally the subject is a domesticated animal.

In one embodiment, the method comprises administration of the taste-masking composition comprising at least one compound that is a purinergic receptor inhibitor in a separate liquid solution or rinse (e.g., mouthwash) in a pharmaceutically acceptable carrier or diluent before exposure to the medicament, nutrient, and/or dietary supplement which comprises an averse tasting API.

In one embodiment, the method comprises administration of the taste-masking composition comprising at least one compound that is a purinergic receptor inhibitor in a dissolving strip or a dissolving film in a pharmaceutically acceptable carrier before exposure to the medicament, nutrient, and/or dietary supplement which comprises an averse tasting API.

In one aspects, provided herein is a regimen for taking a medicament, nutrient, and/or a dietary supplement which comprises an averse tasting API, said regimen comprising administering the taste-masking composition comprising at least one compound that is a purinergic receptor inhibitor, to a patient in a need thereof at least about 1 hour, at least about 30 minutes, at least about 15 minutes, at least about 10 minutes, or at least about 5 minutes prior to the administration of the medicament, nutrient, and/or the dietary supplement.

In another aspect, provided herein is a regimen for taking a medicament, nutrient, and/or a dietary supplement which comprises an averse tasting API, said regimen comprising administering the taste-masking composition comprising at least one compound that is a purinergic receptor inhibitor, with the medicament, the nutrient, and/or the dietary supplement.

In one embodiment, a kit is provided herein comprising an effective amount of a purinergic receptor inhibitor and a pharmaceutically acceptable carrier, as described herein to mix with a medicament, nutrient, and/or a dietary supplement which comprises an aversive tasting API as described herein to suppress aversiveness of the API upon oral administration. In certain embodiments, the kit comprises: (a) multiple thin films (e.g., multiple oral dissolvable films), each in direct contact with at least one other thin film, and each independently described herein; (b) packaging material enclosing the multiple thin films; and (c) printed indicia located on the packaging material; wherein the multiple thin films do not readily stick to another. See also, U.S. Pat. No. 10,307,397 B2.

Conventional methods of measuring and quantifying aversiveness are known to one of skill in the art, e.g., Keast, R. S. J., and Breslin., P. A. S., Modifying the bitterness of selected oral pharmaceuticals with cation and anion series of salts, Pharmaceutical Research, 2002, 19, 1019-1026.

It should be understood that any embodiment of the method or the kit provided herein may be utilized in any other embodiment, composition, method or kit described herein.

The terms “amelioration”, “reduction”, “decrease”, “suppression”, “modulation” or any grammatical variation thereof as used herein may refer to at least about 5%, at least about 10%, at least about 20%, at least about 30%, at least about 40%, at least about 50%, at least about 60%, at least about 70%, at least about 80%, at least about 90%, or at least about 95% of subjects tested showing an intensity of indicated sensation lower than the reference given. In certain embodiment, the terms “amelioration”, “reduction”, “decrease”, “suppression”, “modulation” or any grammatical variation thereof as used herein may refer to an intensity of indicated sensation which is less than about 95%, less than about 90%, less than about 80%, less than about 70%, less than about 60%, less than about 50%, less than about 40%, less than about 30%, less than about 20%, less than about 10%, or less than about 5% of the reference given.

As used herein, the term “blocking” or any grammatical variation thereof may refer to an intensity of indicated sensation which is less than about 96%, less than about 97%, less than about 98%, less than about 99%, or less than about 100% of the reference given.

As used herein, the term “patient” or “subject” as used herein means a mammalian animal, including a human, a veterinary or farm animal, a domestic animal, or pet normally used for clinical research. In one embodiment, the subject of these methods and compositions is a human. In a further embodiment, the subject of these methods and compositions is a child. As used herein, “child” or “children” refers to a human whose age is 0 month to 18 years, including a baby who is 0 to 12 months old; a toddler who is 1 to 3 years old; a preschool child who is 3 to 5 years old; a grade-schooler who is 5 to 12 years old; and a teen who is 12 to 18 years old. In certain embodiments, the subject of these methods and composition is an adult. In yet a further embodiment, the subject of these methods and compositions is a senior adult (also referenced to as “elderly”) who is beyond 65 years old. Still other suitable subjects include, without limitation, non-murine, rat, canine, feline, porcine, bovine, ovine, non-human primate and others. More specifically, as used herein, the term “animal” refers to any non-human animal, including mammals, birds, reptiles, marsupials, amphibians, and fish. In one preferred embodiment, the term “animal” includes domesticated animals, such as a cow, horse, sheep, pig, goat, chicken, turkey, quail, duck, goose, cat, dog, mouse, rat, rabbit, or guinea pig, and is preferably a dog (canine), cat (feline), or horse. The term “animal” also includes wild, non-domesticated animals and exotic animals in captivity, for instance, undomesticated “pets” and animals held in zoological or other captive environments.

As used herein, terms “disease”, “disorder”, and “condition” are used interchangeably, as to indicate an abnormal state in subject.

As used herein, the term “aversiveness” or any grammatical variation thereof refers to an unpleasant sensation selected from the group consisting of bitter taste (bitterness), sourness, astringency, savoriness (umami), and nausea. As used herein, the term “sensation” refers to a perception associated with stimulation of a drug in gastrointestinal tract (e.g., mouth or tongue), including without limitation, taste, astringency and nausea. Conventional methods of quantifying an aversiveness are known to one of skill in the art. See, e.g., Breslin et al, “Suppression of Bitterness by Sodium: Variation Among Bitter Taste Stimuli.” Chemical Senses, Volume 20, Issue 6, 1 Dec. 1995, Pages 609-623, doi.org/10.1093/chemse/20.6.609; Peyrot des Gachons, C. et al, (2011). “Bitter taste induces nausea.” Current Biology, 21, R247-248 PMID 21481757; Keast, R. S. J. and P. A. S. Breslin. (2005) “Bitterness suppression with zinc sulfate and Na-cyclamate: a model of combined peripheral and central neural approaches to flavor modification”. Pharmaceutical Science, 22, 1970-1977. PMID: 16132352; Breslin PA, and Tharp CD. “Reduction of saltiness and bitterness after a chlorhexidine rinse”, Chem Senses. 2001 Feb;26(2):105-16; and Keast, R. S. J. and P. A. S. Breslin. (2002) “Modifying the bitterness of selected oral pharmaceuticals with cation and anion series of salts.” Pharmaceutical Research, 19, 1019-1026. PMID: 12180534, each of which is incorporated herein in its entirety.

As used herein, the term “administration” or any grammatical variations thereof refers to delivery of composition described herein to a subject. “Oral administration” or any grammatical variations thereof refers to an administration where composition described herein is taken by the subject through mouth. In one embodiment, oral administration may include without limitation, enteral administration, wherein the composition is taken through mouth and absorbed in the gastrointestinal tract; buccal administration wherein the composition is dissolved inside the cheek; sublabial administration, wherein the composition is dissolved under the lip; and sublingual administration, wherein the composition is dissolved under the tongue. In certain embodiments, the oral administration includes topical oral administration the composition is dissolved in an epithelium of oral cavity to penetrate epithelium deeply enough to affect taste nerves. Furthermore, the form of administration can be liquid (i.e., solutions or suspensions), soluble or dispersible tablets, oral wafers, chewable tablets, orodispersible tablets, dissolving strip, coating applied on a food product, coating applied on an API, chewable food product, chewable or rapid dissolve tablet, oral topical spray, oral topical mist that coats epithelium, a lollipop, or a lozenge.

As used herein, “an effective amount” refers to a concentration of a compound that is a purinergic receptor inhibitor in the formulation of a composition, a concentration of a compound that is a purinergic receptor inhibitor in mouth, or a molar ratio of a medicament (or a nutrient, or a dietary supplement comprising averse tasting API) to a compound that is a purinergic receptor inhibitor, which suppresses aversiveness of the orally administrated medicament compared to the oral administration of the medicament only. In one embodiment, “an effective amount” might also refer to an amount of a compound that is a purinergic receptor inhibitor when mixed with a medicament and administrated orally, suppresses aversiveness of the API in a medicament.

In certain embodiments, the effective amount is defined as the molar ratio of API (or drug, medicament, nutrient or dietary supplement) to the at least one compound that is purinergic receptor inhibitor, which is about 0.00003 (3×10⁻⁵) to about 1.0, or any number including and between these numbers. In one embodiment, the effective amount is a molar ratio of the API/medicament to the purinergic receptor inhibitor is about 0.0001 (1×10⁻⁴) to about 0.0010 (1×10⁻³) or any number including and between these numbers. In another embodiment, the effective amount is a molar ratio of the API/medicament to the purinergic receptor inhibitor is about 0.001 (1×10⁻³) to about 0.010 (1×10⁻²) or any number including and between these numbers. In yet another embodiment, the effective amount is a molar ratio of the API/medicament to the purinergic receptor inhibitor is about 0.01 to about 0.10 or any number including and between these numbers. In a further embodiment, the effective amount is a molar ratio of the API/medicament to the purinergic receptor inhibitor is about 0.1 to about 0.5 or any number including and between these numbers. In yet further embodiment, the effective amount is a molar ratio of the API/medicament to the purinergic receptor inhibitor is about 0.5 to about 1.0 or any number including and between these numbers.

As used herein, the term “salt” refers to a chemical compound consisting of one, two or more positively charged cation(s) and one, two or more negatively charged anion(s). In some embodiments, the cation is selected from the group consisting of H⁺, Na⁺, K⁺, Ca²⁺, Cs⁺, and Zn²⁺. In some embodiments, the anion is selected from the group consisting of Cl—, Gluconate, Glutamate, Adenosine Monophosphate, Phosphatidate, Diphosphates, Phosphate, Citrate, Malate, Tartarate, Ascorbate, and Hydroxide.

As used herein, the terms “drug” “pharmaceutical drug” and “pharmaceuticals” are used interchangeably and refer to a composition comprising a chemical or biological compound which has a physiological effect when administrated in a subject in need (e.g., the active pharmaceutical ingredient or API), and a pharmaceutical acceptable carrier. In certain embodiments, the term “drug” refers to an API. In certain embodiments, the terms “medicament”, “nutrient”, and “dietary supplement” refers to a drug including an API. In other embodiments, in which aversion is caused by a component of the pharmaceutical composition other than the API, the term “drug” encompasses any ingredient of the composition, including but not limited to the API. As used herein, a physiological effect refers to stopping or reversing progression of a disease (e.g., infection with bacteria, plasmodia, and helminths). The physiological effect might include but not limited to clearing a parasite, a bacterium, a fungal, or a virus, from the subject.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language. As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an” refers to one or more. As such, the terms “a” (or “an”), “one or more,” and “at least one” are used interchangeably herein.

As used herein, the term “about” or “˜” refers to a variant of 10% from the reference integer and values therebetween, unless otherwise specified. For example, “about” 500 μM includes ±50 (i.e., 450-550, which includes the integers therebetween). For other values, particularly when reference is to a percentage (e.g., 90% of taste), the term “about” is inclusive of all values within the range including both the integer and fractions.

As used herein, the term “about” means a variability of plus or minus 10% from the reference given, unless otherwise specified.

In certain instances, the term “E+#” or the term “e+#” is used to reference an exponent. For example, “5E10” or “5e10” is 5×10¹⁰. These terms may be used interchangeably.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### Examples

The following examples are provided to illustrate certain aspects of the claimed invention. The invention is not limited to these examples.

Purinergic receptor inhibitors were developed for treating diseases such as cough, inflammation, arthritis, inflammatory bowel syndrome pain) after systemic dosing. See also, Abdulgawi, R., et al., P2X3 receptor antagonist (AF-219) in refractory chronic cough: a randomized, double-blind, placebo-controlled phase 2 study, Lancet, 2015, 385: 1198-1205, ePub Nov. 25, 2014; Ford, A. P., In pursuit of P2X3 antagonists: novel therapeutics for chronic pain and afferent sensitization, Purinergic Signaling, 2012, 8(1):S3-S26, ePub Nov. 18, 2011; Gever, J. R., et al., AF-353, a novel, potent and orally bioavailable P2X3/P2X2/3 receptor antagonist, Br J Pharmacol 2010 Jul; 160(6): 1387-1398; and Marucci, G., et al., Update on novel purinergic P2X3 and P2X2/X3 receptor antagonist and their potential therapeutic applications, Expert Opinion on Therapeutic Patents, 2019, 29(12): 943-963, ePub Nov. 14, 2019, which are all incorporated herein by reference in its entirety.

Taste side effects are known to occur in humans who systemically dose, but the idea of blocking taste with an oral rinse of P2×3 blockers is unknown in the medical literature.

We tested a potent and specific P2X3 receptor inhibitor as a taste blocker in humans following a topical rinse and expectoration of the drug. There are two specific drugs we tested that are P2X3 receptors, both made by Roche: Roche AF-353 (P2X3 inhibitor), Roche AF-219 (P2X3 inhibitor). In addition, we use a negative control drug that does not work, that is a specific P2X7 inhibitor made by AstraZeneca: AstraZeneca AZD-9056 (P2X7 inhibitor). The logic behind our strategy is that all taste cells in taste buds, which are not neurons, must communicate with neurons in and around the taste bud to send taste signals to the brain. The chemical signal that taste bud cells use is adenosine triphosphate (ATP), a purine. Neurons receive this signal when ATP binds to the neural purinergic receptor, i.e., P2X3. There are many different purinergic receptors, but taste appears to primarily use P2X3. There is evidence that painting the P2X3 inhibitor AF-353 onto the tongues of mice affects their ability to taste behaviorally and electro-physiologically. See also, Vandenbeuch, A., et al., Postsynaptic P2X3-containing receptors in gustatory nerve fibers mediate responses to all taste qualities in mice, J Physiol. 2015 Mar. 1; 593(Pt 5): 1113-1125. Published online 2015 Jan 20, which is incorporated herein by reference in its entirety.

In this study, we observed that composition comprising an effective amount of AF-353 completely blocked taste (sweet, sour, salty, bitter (Quinine and Praziquantel)), but did not block aroma or carbonation irritation in 4 people. In some experiments, we used a regimen of two 1-minute rinses with 500 μM AF-353 composition (i.e., oral topical administration). We observed that at some concentrations the blockade was not an immediate blockade, and required approximately 5 to 10 minutes of wait time before the taste was blocked. We additionally observed that recovery of the ability to taste began within 60 minutes, and the ability to taste fully recovered after 1.5-3 hours.

We examined this invention in humans using a solubilization formula and shown that topical oral rinses are relatively fast acting (i.e., 2 minutes), highly effective (total taste blockade), relatively short lived (one to two hours), all without the need for swallowing or systemic dosing, and without negative side effects such as oral numbing, tingling, or strange oral sensations. This was not previously known to work in humans, to be effective quickly, to penetrate highly cornified oral epithelial tissue rapidly, to return to normal within one to two hours, or to have such a highly specific effect on taste blockade without affecting other oral sensory modalities.

Additionally, we examine further specifics on both concentration (dose) and timing of treatment as a regimen to allow for minimal dose with maximal effect achieved. We examine ratios of aversiveness of target to concentration of taste blockade treatment, parallel as to how pain medications are dosed in a ratio of drug dose to pain level being treated.

### EXAMPLE 1—Block in Taste Following Topical Oral Administration of a Composition Comprising AF-353

In this study, we tested an AF-353 composition in humans as an oral rinse after receiving IRB approval for its use. First, we tested rinsing 100 μM (0.04 mg/mL) orally for 2 minutes as 4×30 sec rinses with expectoration. We tested before and after sour, bitter, sweet, and salty solutions (quinine HCl, NaCl, Praziquantal, sucrose, CO2). All solutions were moderate in intensity before treatment and were mild in intensity after treatment in four participants. We found that the impact of the drug at reducing taste increased further at 10 to 15 minutes after we completed rinsing. This may be due to the drug penetrating deeper into the epithelium to the taste nerves.

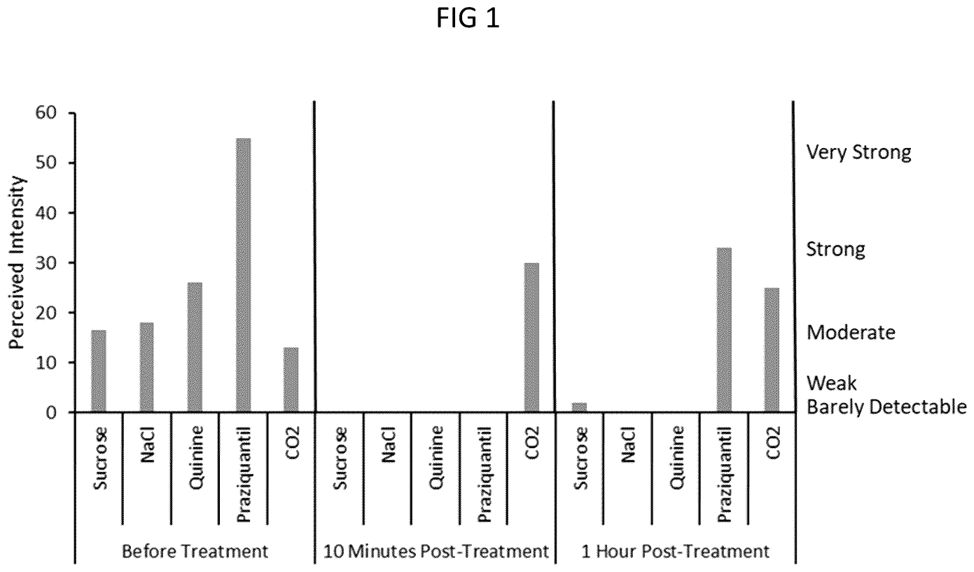

Next, we tested rinsing 500 μM (0.2 mg/mL) AF-353 for two minutes, in 2×1 min increment rinses with expectoration. All tastes were eliminated. Recovery from this abolishment of taste was at approximately 50 to 75% of normal at 1 hour and was 100% of normal at 2 hours after rinsing with AF-353. Additionally, we have preliminary tested a treatment composition comprising AF-219 at a 500 μM, which is also a P2X3 inhibitor, and found that it did not block taste at this concentration. FIG. 1 shows a graph representing perceived intensities of taste and chemesthetic stimuli before and after treatment with AF-353 (n=4) oral rinse composition in humans. The y-axis represents perceived intensity ratings on a labeled magnitude scale. The x-axis represents taste and chemesthetic stimuli including sucrose, NaCl, quinine, the pharmaceutical Praziquantil, and carbonated water with lime aroma. The first 5 bars on the left are ratings of intensity before treatment. The next five bars are ratings of intensity after two 1-minute rinses with 500 μM AF-353. The last five bars on the right are ratings of intensity 1-hour post treatment with AF-353. In some subjects, the recovery of taste perception was about 20 minutes faster. Furthermore, we tested AZD-9056, which is P2X7 inhibitor and used as a negative control, at various concentration in a control composition and found that it did not block taste, consistent with known taste physiology.

Furthermore, additional doses are tested to identify effective dose of AF-353 and AF-219, and to refine the sensory protocol. In a pre-taste test step, the sour, bitter, sweet, and salty solutions (quinine HCl, NaCl, Praziquantal, sucrose, C02) including tenofovir alafenamide (TAF), which is an anti-viral medicament, are tasted, expectorated, and rated in intensity of the taste stimuli. Then, in a treatment step, subjects are administered with topical oral rinse of about 10 mL treatment composition at various concentrations including ranging between 50 μM to 1000 μM, in which the treatment composition (solution) comprises AF-353, and the control composition (solution) comprises AF-219. Next, in a post-taste step, the sour, bitter, sweet, and salty solutions (quinine HCl, NaCl, Praziquantal, sucrose, C02) including tenofovir alafenamide (TAF), are again tasted, expectorated, and rated in intensity of the taste stimuli.

### EXAMPLE 2—Optimization of a Formulation of AF-353 for Administration in Human Subject to Facilitate Compliance of Taking and Increase in Taking a Medicament with Negative Taste Effects

One of the challenges of utilizing purinergic receptor inhibitors, i.e., P2X3 inhibitor, or AF-353, is that these inhibitors tend to be lipophilic, and therefore may not go into solution easily. In mice one can use DMSO to treat the mouth with lipophilic drugs. However, such approach cannot be done for administration of the inhibitor in human subjects. It is also unclear if the drug would penetrate deeply enough into the epithelium to affect taste nerves. Furthermore, there is evidence that purinergic receptor inhibitors can block a variety of sensory signals, so the drug may have induced numbness or a variety of strange and unpleasant sensations. However, as described in a study of Example 1, the treatment was tasteless and did not cause numbness or any strange sensations, only taste blockade. Thus, we are first to show the specific manner of treatment and solubilization (e.g., with ethanol and tween), the concentrations of use, and the timing of treatment which were unknown until now. These P2X3 inhibitors could be used to make an aversive medical oral treatment with negative taste side effects tolerable such as anti-malarial, anti-HIV, and anti-helminth drugs in children's formulations. Use of these taste blockers prior or with administration of medicament with negative taste side-effects could allow for facilitating adherence and overall compliance to medicament regimen in children, which is especially important in combating viral diseases. In addition, nutrients such as essential amino acids that elderly are often asked to ingest to combat muscle wasting (sarcopenia) can be difficult to tolerate due to bitterness. These taste blockers could allow elderly to ingest their prescribed amino acid medical treatments to combat muscle wasting.

In one aspect, the taste blocking composition could be provided to children in the form of a small lollipop (sucker) or a dissolving strip with color (e.g., purple) and flavor (e.g., grape) with just the right amount of sugar to time the treatment and just the right dosses to be effective at blocking the aversive taste of drugs. Children could do this even while waiting in line for treatment as long as 30 minutes prior to medicament dosing. For elderly, this could be used the same as with children or they could have an oral spray or rapid dissolve film or tablet or chewable prior to dosing with aversive tasting medicament or nutrient.

In this study, we further explore the dose efficacy, the timing of treatment with taste blocking composition, and the mode of delivery, such as in a gummy or lollipop with any eye towards minimal effective dose and brevity of treatment timing. Additionally, we explore the efficacy of treatment on the very high levels of bitterness. Furthermore, we determine the specificity to taste by examining effects on astringents, irritants, and “metallic” tastes.

### EXAMPLE 3—Investigation of P2X2/P2X3 Antagonists as Bitter Blockers—Background and Experimental Procedures

### Background

AF-353 (5-(5-iodo-2-isopropyl-4-methoxy-phenoxy)-pyrimidine-2,4-diamine) AF-353 is a dual P2X3/P2X2/3 receptor antagonist. AF-353 has high oral bioavailability, a half-life of 1.63 h (in rat) and good CNS penetration in addition to high P2X3/P2X2/3 antagonist potency [Gever, J. R.; et al., AF-353, a novel, potent and orally bioavailable P2X3/P2X2/3 receptor antagonist. Br. J. Pharmac 160:1387-1398; 2010]. Solutions (2 mg/mL) and suspensions of AF-353 in acidic media are physically and chemically stable for at least 4 weeks at room temperature [Gever, J. R., et al., AF-353, a novel, potent and orally bioavailable P2X3/P2X2/3 receptor antagonist. Br. J. Pharmac 160:1387-1398; 2010]. AF-353 has been reported to attenuate the pain caused by bone cancer in rats [Kaan, T. K., et al., Systemic blockade of P2X3 and P2X2/3 receptors attenuates bone cancer pain behaviour in rats. Brain 133:2549-2564; 2010]. AF-353 also counteracts activity mediated by P2X3/P2X2/3 receptors on bladder afferent nerves of rats [Ferguson, A. C., et al., Inhibition of urothelial P2X3 receptors prevents desensitization of purinergic detrusor contractions in the rat bladder. BJU Int 116:293-301; 2015; Kaan, T. K., et al., Endogenous purinergic control of bladder activity via presynaptic P2X3 and P2X2/3 receptors in the spinal cord. J Neurosci 30:4503-4507; 2010; Munoz, A., et al., Modulation of bladder afferent signals in normal and spinal cord-injured rats by purinergic P2X3 and P2X2/3 receptors. BJU Int 110:E409-414; 2012; Salazar, B. H., et al., A. Electrical activity of the bladder Is attenuated by intravesical inhibition of P2X2/3 receptors during micturition in female rats. Int Neurourol J 21:259-269; 2017; Salazar, B. H., et al., Modulatory effects of intravesical P2X2/3 purinergic receptor inhibition on lower urinary tract electromyographic properties and voiding function of female rats with moderate or severe spinal cord injury. BJU Int 123:538-547; 2019]. AF-353 blocks bronchoconstriction-induced nodose C-fiber discharge in guinea pigs [Weigand, L. A., et al., A role for ATP in bronchoconstriction-induced activation of guinea pig vagal intrapulmonary C-fibres. J Physiol 590:4109-4120; 2012].

AF-219 (Gefapixant; MK-7264; C14H19N504S; MW=approx. 344; 1 pill=0.131 mmol) has been approved as a treatment of refractory common cough [Cui, W. W., et al., P2X3-selective mechanism of Gefapixant, a drug candidate for the treatment of refractory chronic cough. Comput Struct Biotechnol J 20:1642-1653; 2022]. AF-219 at a dose of 600 mg twice daily caused “clinically significant” taste disturbances in all patients tested [Abdulgawi, R., et al., P2X3 receptor antagonist (AF-219) in refractory chronic cough: a randomized, double-blind, placebo-controlled phase 2 study. Lancet 385:1198-1205; 2015]. It has been reported that doses as low as 7.5-50 mg twice daily also cause taste disturbances, albeit less severe [Sheridan, C. Merck stakes out ‘irritable’ neuron territory with $1.25 billion. Nature biotechnology 34:900; 2016].

Additionally, it has been previously reported that BLU-5937 (Methyl (S)-3-((2-(2,6-difluoro-4-(methylcarbamoyl)phenyl)-7-methylimidazo[1,2-a]pyridin-3-yl)methyl)piperidine-1-carboxylate; 10-20 mg/kg, intraperitoneally (ip)) in rat did not influence taste whereas N-00588 did [Garceau, D.; Chauret, N. BLU-5937: A selective P2X3 antagonist with potent anti-tussive effect and no taste alteration. Pulm Pharmacol Ther 56:56-62; 2019]. Groups of 10 thirsty rats were administered ascending daily doses of BLU-5937 ip or N-00588 ip (in a 10% PG in saline vehicle) and then received a 15-min two-bottle choice test with 0.3 mM quinine hydrochloride and water in the bottles. The rats avoided the quinine after injection of BLU-5937 (10 or 20 mg/kg) but drank it after N00588 (10 or 20 mg/kg).

“The high selectivity of BLU-5937 for homotrimeric receptors sets it apart from other P2X3 antagonists.” Afferent nerve fibers innervating taste buds stain for P2X2 and P2X3 subunits [Bo, X., et al., Localization of ATP-gated P2X2 and P2X3 receptor immunoreactive nerves in rat taste buds. Neuroreport 10:1107-1111; 1999]. P2X2 and P2X3 subunits are co-localized on gustatory nerves [Ishida, Y., et al., P2X(2)—and P2X(3)-positive fibers in fungiform papillae originate from the chorda tympani but not the trigeminal nerve in rats and mice. J. Comp. Neurol. 514:131-144; 2009]. Mice with double KO of P2X2 and P2X3 have dysfunctional taste responses whereas KO of either channel alone has minimal effects on taste [Cockayne, D. A, et al., P. P2X2 knockout mice and P2X2/P2X3 double knockout mice reveal a role for the P2X2 receptor subunit in mediating multiple sensory effects of ATP. J Physiol 567:621-639; 2005; Finger, T. E., et al., ATP signaling is crucial for communication from taste buds to gustatory nerves. Science 310:1495-1499; 2005].

In one clinical trial, eliapixant (BAY1817080), a selective P2X3 antagonist, caused taste-related adverse events [primarily dysgeusia; (Morice, A., et al, Eliapixant (BAY 1817080), a P2X3 receptor antagonist, in refractory chronic cough: a randomised, placebo-controlled, crossover phase 2a study. Eur Respir J 58; 2021)] although it had no concerted effects on ratings of taste intensity measured using taste strips [Klein, S., et al., First-in-human study of eliapixant (BAY 1817080), a highly selective P2X3 receptor antagonist: tolerability, safety and pharmacokinetics. Br. J. Cli. Pharmac.; 2022].

Mice that cannot release ATP from taste cells due to the genetic deletion of CALHM1 or CALHM3 [Ma, Z., et al., CALHM3 is essential for rapid ion channel-mediated purinergic neurotransmission of GPCR-mediated tastes. Neuron 98:547-561 e510; 2018; Taruno, A, et al., CALHM1 ion channel mediates purinergic neurotransmission of sweet, bitter and umami tastes. Nature 495:223-226; 2013; Tordoff, M. G., et al., Salty taste deficits in CALHM1 knockout mice. Chem. Senses 39:515-528; 2014], or that cannot detect extracellular ATP in gustatory afferent nerves due to the genetic deletion of both P2X2 and P2X3 [Cockayne, D. A., et al., P2X2 knockout mice and P2X2/P2X3 double knockout mice reveal a role for the P2X2 receptor subunit in mediating multiple sensory effects of ATP. J Physiol 567:621-639; 2005; Eddy, M. C., et al., Double P2X2/P2X3 purinergic receptor knockout mice do not taste NaCl or the artificial sweetener SC45647. Chem. Senses 34:789-797; 2009; Finger, T. E., et al., ATP signaling is crucial for communication from taste buds to gustatory nerves. Science 310:1495-1499; 200] are “taste blind”-they show few-or-no responses to taste stimuli, and responses that do persist can be attributed to non-taste mechanisms. For example, P2X2/P2X3 double knockout mice show diminished but not complete avoidance of high concentrations of bitter compounds (caffeine and quinine) and acids (sour tastes) but the residual responses can be accounted for by irritation of the oropharynx, upper airway, or upper gastrointestinal tract [Freund, J. R.; Lee, R. J., Taste receptors in the upper airway. World J Otorhinolaryngol Head Neck Surg 4:67-76; 2018; Hallock, R. M., et al., Residual chemosensory capabilities in double P2X2/P2X3 purinergic receptor null mice: intraoral or postingestive detection? Chem. Senses 34:799-808; 2009] or by direct involvement of nerve fibers innervating the laryngeal epithelium [Hallock, R. M., et al., Residual chemosensory capabilities in double P2X2/P2X3 purinergic receptor null mice: intraoral or postingestive detection? Chem. Senses 34:799-808; 2009; Ohkuri, T., et al., Residual chemoresponsiveness to acids in the superior laryngeal nerve in “taste-blind” (P2X2/P2X3 double-KO) mice. Chem. Senses 20:523-532; 2012]. Similarly, CALHM1 and P2X2/P2X3 knockout mice show preferences for monosodium glutamate, sucrose, maltodextrin and fat but these are observed only if the mice have had prior experience with these nutrients [Hallock, R. M., et al., Residual chemosensory capabilities in double P2X2/P2X3 purinergic receptor null mice: intraoral or postingestive detection? Chem. Senses 34:799-808; 2009; Sclafani, A.; Ackroff, K. Maltodextrin and fat preference deficits in “taste-blind” P2X2/P2X3 knockout mice. Chem. Senses; 2014; Sclafani, A.; Marambaud, P.; Ackroff, K. Sucrose-conditioned flavor preferences in sweet ageusic T1r3 and Calhml knockout mice. Physiol Behav 126:25-29; 2014; Stratford, J. M.; Finger, T. E. Central representation of postingestive chemosensory cues in mice that lack the ability to taste. J Neurosci 31:9101-9110; 2011], allowing flavor-nutrient learning to occur [Tordoff, M. G. Metabolic basis of learned food preferences. In: Friedman, M. I.; Kare, M. R.; Tordoff, M. G., eds. Chemical senses: appetite and nutrition. Vol. 4. New York: Marcel Dekker; 1991:239-260]. Although, the off-taste of high concentrations of artificial sweeteners can be attributed to activation of TRPV1 channels [Riera, C. E., et al., The capsaicin receptor participates in artificial sweetener aversion. Biochem. Biophys. Res. Comm. 376:653-657; 2008].

Vandenbeuch et al [Vandenbeuch, A., et al., Postsynaptic P2X3-containing receptors in gustatory nerve fibres mediate responses to all taste qualities in mice. J Physiol 593:1113-1125; 2015] found that, in anesthetized mice, 1.0 or 1.1 mM AF-353 flowed over the anterior tongue for 10 min eliminated chorda tympani nerve responses elicited by orally applied taste stimuli at 10 and 30 min after application [Larson, E. D., et al., Function, innervation, and neurotransmitter signaling in mice lacking Type-II taste cells. eNeuro 7; 2020; Vandenbeuch, A., et al., Postsynaptic P2X3-containing receptors in gustatory nerve fibres mediate responses to all taste qualities in mice. J Physiol 593:1113-1125; 2015]. Intraperitoneal injection of a high dose of AF-353 (6 mg/kg; plasma concentration 44 μM) abolished the chorda tympani nerve responses to all tastants; a dose of 0.25 mg/kg (plasma concentration 1.3 μM) decreased responses by ˜50%. These authors also measured the licking behavior of mice to the artificial sweetener SC-45647 after intraperitoneal administration of AF-353 or vehicle (10% propylene glycol). They found that the preference for SC-45647 shown by vehicle-administered mice was eliminated in AF-353 treated mice. In two-bottle choice tests, P2X3-only KO animals were significantly impaired in their responses to SC45647, saccharin, and denatonium, and P2X2-only KO mice were marginally impaired to denatonium.

### Experimental Procedures

General procedures

The methods were adapted from those described by Vandenbeuch et al [Vandenbeuch, A., et al., Postsynaptic P2X3-containing receptors in gustatory nerve fibres mediate responses to all taste qualities in mice. J Physiol 593:1113-1125; 2015] who, (a) in anesthetized mice, flowed AF-353 over the anterior tongue then recorded gustatory nerve responses to tastants [see also (Larson, E. D., et al., Function, innervation, and neurotransmitter signaling in mice lacking Type-II taste cells. eNeuro 7; 2020)] and (b), in awake mice, injected AF-353 intraperitoneally then measured licking for the sweetener SC-45647. Here, we applied AF-353 to the oral cavity of briefly anesthetized mice then measured licking using procedures similar to those of our past work [Med Associates inc. Products: Gustometry, med-associates.com/product/davis-rig-for-mouse-16-bottle/. 2022; Taruno, A., et al., CALHM1 ion channel mediates purinergic neurotransmission of sweet, bitter and umami tastes. Nature 495:223-226; 2013; Tordoff, M. G., et al., Normal taste acceptance and preference of PANX1 knockout mice. Chem. Senses 2015 May 18. pii: bjv025; 2015; Tordoff, M. G.; Aleman, T. R.; McCaughey, S. A. Heightened avidity for trisodium pyrophosphate in mice lacking Taslr3. Chem. Senses 40:53-59; 2014; Tordoff, M. G.; Ellis, H. T. Taste dysfunction in BTBR mice due to a mutation of Itpr3, the inositol triphosphate receptor 3 gene. Physiol. Genomics 45:834-855; 2013] and other investigators [Glendinning, J. I.; Gresack, J.; Spector, A. C. A high-throughput screening procedure for identifying mice with aberrant taste and oromotor function. Chem. Senses 27:461-474; 2002].

Groups of 16 male C57BL/6J mice aged 7 weeks old were purchased from The Jackson Laboratory (strain ID number 000664). They were maintained in a vivarium at 23° C. on a 12 h:12 h light/dark cycle, with lights off at 1800. When not being tested, mice were housed alone in plastic tub cages (26.5×17×12 cm) with a stainless-steel grid lid and wood chips scattered on the floor [see (Tordoff, M. G.; Bachmanov, A. A. Monell mouse taste phenotyping project. Monell Chemical Senses Center, monell.org/MMTPP. 2001) for details]. The mice ate pelleted chow (Teklad 8601) and drank deionized water according to the regimen described below. Each mouse was weighed daily, immediately before it was placed into a gustometer.

Brief-exposure taste acceptance was assessed using MS160-Mouse gustometers manufactured by DiLog Instruments (now available from Med Associates). Each gustometer consists of a 14.5×30×15 cm test chamber with a motorized shutter that controls access to a taste solution. Bottles of taste solution are mounted on a rack that is precisely positioned by a stepper motor so that any one of eight different taste solutions can be presented to the mouse. The drinking spout of each bottle is part of a high-frequency alternating current contact circuit, so that each lick the mouse makes is detected and recorded. Details of construction and other technical information are available elsewhere [Med Associates inc. Products: Gustometry, med-associates.com/product/davis-rig-for-mouse-16-bottle/. 2022; Sheridan, C. Merck stakes out ‘irritable’ neuron territory with $1.25 billion. Nature biotechnology 34:900; 2016].

To train the mice to sample taste solutions, they were first deprived of water for 22.5 h, and then placed into a gustometer with its shutter open. During this first training session, each mouse had continuous access to water for 30 min. It was then returned to its home cage and given water for 1 h. During the following two days, this procedure was repeated, except the shutter allowing access to water was closed 5 sec after each time the mouse began to lick, and was re-opened after a 7.5-sec interval. After 20 min, the mouse was returned to its home cage and given water for 1 h. By the second test using these procedures, nearly all mice had learned to obtain water during the 5-sec access periods. If a mouse failed to lick by the second session all 16 mice in the cohort were given a third day of training.

### Test Procedure

Tests were conducted once a day, generally on four successive days, in the middle of the light period. Each water-deprived mouse was anesthetized by placing it in a sealed plastic cage with an acrylic lid. On the floor of the cage was an isoflurane-soaked gauze pad. Care was taken to ensure the mouse did not come into direct contact with the isoflurane. When the mouse was unconscious, its oral cavity was swabbed repeatedly with a wooden handled cotton swab (a cotton-tipped applicator; Henry Schein). The swab was modified such that ˜75% of the cotton tip was removed by unwrapping it, leaving cotton in a cone, similar in shape to a pencil. The cotton tip was dipped in a solution of AF-353 or its vehicle (DMSO). To prepare the AF-353, 10 mg powder purchased from Cayman Chemical (catalog no. 23034) was first dissolved in 1 mL DMSO (Sigma-Aldrich; catalog no. D8418) to make a 25-mM stock solution; this was then diluted with deionized water to a volume of ˜11 mL (i.e., sufficient 3 mM AF-353 for the day's test of 8 mice). Care was taken to brush the cotton swab over the dorsal and ventral surfaces of the tongue and the buccal mucosa. Approximately every minute, as needed, the mouse was re-exposed for a few seconds to an isoflurane-soaked gauze pad to keep it anesthetized. The duration of AF-353 treatment was monitored with a stopwatch and unless otherwise mentioned was 4 min.

Subsequent procedures depended on which of two series of tests was being conducted: Determining the optimal parameters to observe an effect of AF-353 on bitter taste. In this series of experiments, the goal was to discover the optimal duration of treatment with AF-353, the optimal concentration of AF-353, and the optimal time between AF-353 treatment and testing.

For each experiment, each mouse was tested four times over four successive days, once in each condition, according to a counterbalanced design. In the experiment designed to assess optimal duration, the time AF-353 was applied to the oral cavity was set at 0 (no AF-353 was applied), 2, 4 or 8 min. In the experiment designed to assess optimal concentration, the concentration of AF-353 applied to the oral cavity was 0 (i.e., DMSO vehicle), 0.3, 1 or 3 mM. Based on the change in weight of cotton-tipped applicators before and after swabbing the oral cavity, we calculate that ˜135 μl of the AF-353 solution soaked into a cotton swab was deposited in the oral cavity, so the oral doses of AF-353 were 0.04, 0.12 and 0.41 μmol/mouse. The mice weighed (mean±SEM) 22.0±0.4 g, so doses in relation to body weight (BW) were 1.8, 6 or 18 nmol/g BW or 0.72, 2.4 and 7.2 mg/kg BW.

Two experiments were designed to determine the duration of action of AF-353. In one, the time between the end of AF-353 treatment and beginning of testing was set at 0, 5, 10 or 20 min. However, it was clear from observation of the mice while they were in the gustometer that they were still groggy and incapacitated from the isoflurane at 0 and 5 min after treatment. Consequently, the results of this experiment are not presented. In the other experiment, the time between the end of AF-353 treatment and beginning of testing was set at 10, 30, 60 or 90 min.

In one experiment, we compared the effect of AF-353 administered to the oral cavity with the same dose of AF-353 administered by intraperitoneal (IP) injection. This experiment employed a 2×2×2 completely within-subjects design, with factors of route of administration (oral swabbing or IP injection), AF-353 (vehicle or 3 mM [0.41 μmol/mouse] AF-353) and fluid licked (water or 1 mM quinine hydrochloride (QHCl)). Before both the oral application and the IP injection, mice were anesthetized with isoflurane. For the oral condition, AF-353 (3 mM; 0.41 μmol/mouse) was swabbed on the oral cavity for 4 min and the mouse was tested 10 min later; for the IP condition, an injection of 0.41 μmol AF-353 was given 2 min after anesthesia began, 2 min later anesthesia was discontinued and the mouse was tested 10 min later.

In all these experiments, the vehicle for AF-353 was Dimethyl sulfoxide (DMSO) but in related studies involving measurement of the effect AF-353 rinses on taste in humans, the vehicle was grain alcohol and Tween 80. The final experiment of this series directly compared the effect of AF-353 in a vehicle of DMSO with AF-353 in a vehicle of alcohol and Tween. The experiment involved a 2×2×2 completely within-subjects design with factors of vehicle (DMSO or alcohol+Tween), AF-353 (vehicle or 3 mM), and fluid licked (deionized water or 1 mM QHCl). For the DMSO condition the AF-353 was prepared as in previous experiments (AF-353 was dissolved in 1 mL DMSO to make a 25-mM stock solution). To prepare AF-353 in alcohol+Tween, 50 μl of 75.5% grain alcohol (Everclear brand) was added to 10 mg of AF-353, then 950 μl of 1% Tween-80 solution was added, to make a 25 mM AF-353 solution. This did not fully dissolve, so an additional 400 μl alcohol and 4 μl Tween-80 were added, yielding a 17.86 mM AF-353 stock solution. The stock solutions were then diluted with deionized water to prepare 3 mM solutions that were swabbed onto the oral cavity. Vehicle only stock solutions were prepared the same way, but without the AF-353.

Each mouse was tested in a gustometer with alternating 10-sec access periods to deionized water or 1 mM quinine hydrochloride (QHCl), with 7.5 sec between each access period. The cycle of 10-sec water-7.5-sec interval-10-sec QHCl was repeated for 15 min, although most mice licked only during the first few minutes of the test. After the session, each mouse received water for 1 h in its home cage and it was then deprived of water in preparation for the next day's session.

### Determining the Effect of AF-353 on Medicines and Exemplars of Basic Tastes

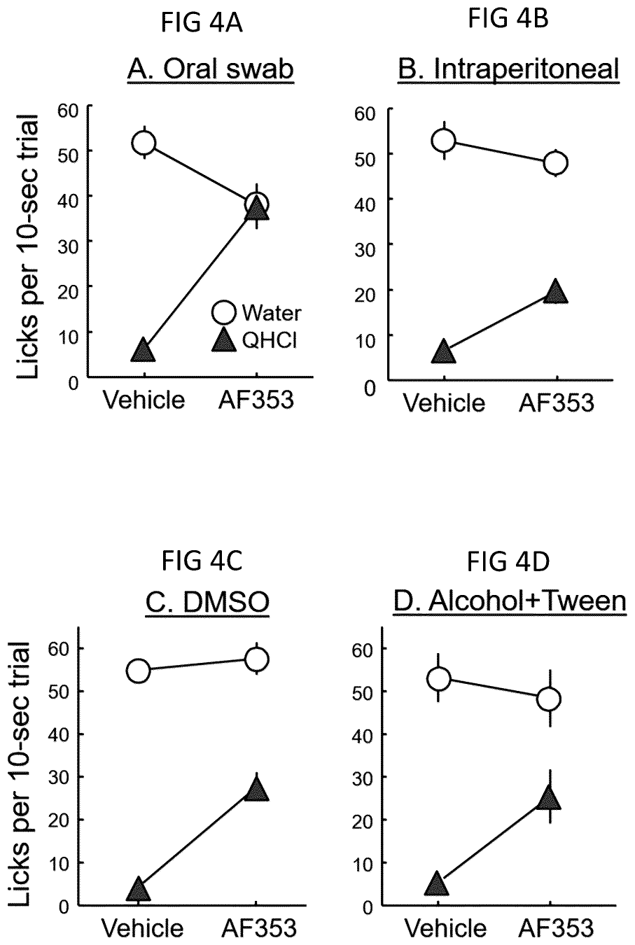

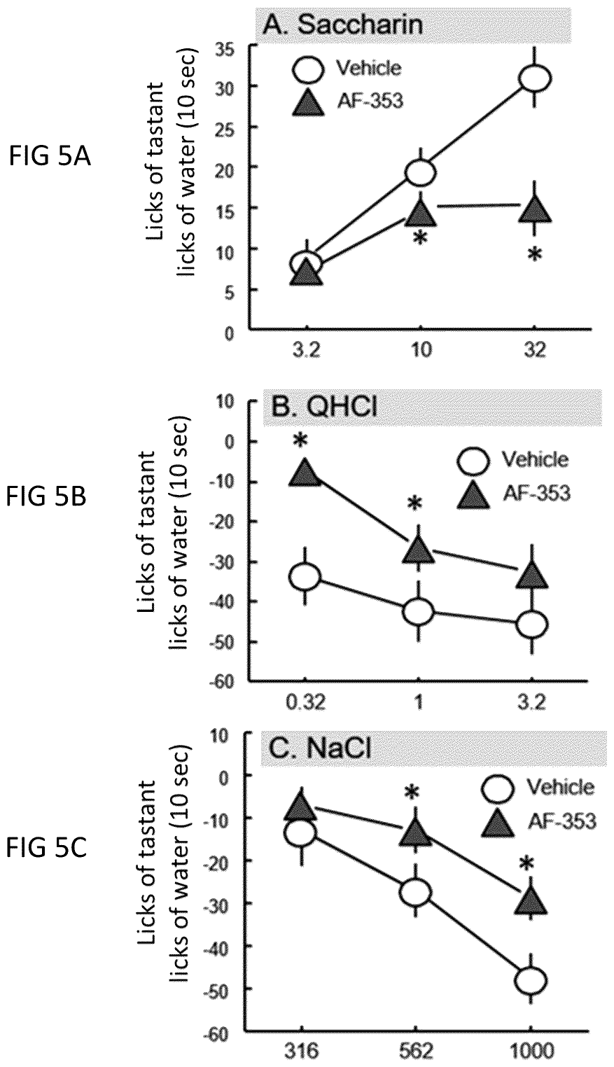

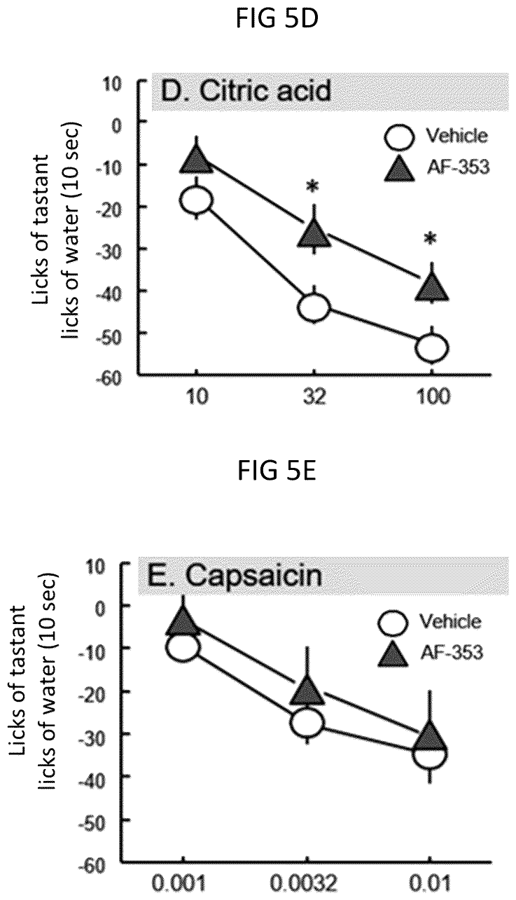

The goal of this series of experiments was to assess the generality of taste loss caused by AF-353. Cohorts of 16 mice were tested four times over four days. Each mouse was tested twice after application of 3 mM AF-353 and twice after application of DMSO, with treatments presented in a counterbalanced order. Each mouse was anesthetized with isoflurane, treated with 3 mM AF-353 or DMSO for 4 min, and tested 10 min later in a gustometer. In each experiment, deionized water and three concentrations of various tastants were presented; each tastant was tested in a separate experiment. (We use the word “tastant” here for convenience, to mean whatever we gave the mice to lick: some of the compounds have odors and/or trigeminal effects and so are not just tastes). The concentrations of each tastant tested are shown in [FIGS. 4A to 4C, and FIGS. 5A to 5E]. They were chosen to span the range from hedonic indifference to almost total rejection based on previous literature or pilot experiments (except for saccharin, which spanned the range from hedonic indifference to strong acceptance; see below).

Most taste compounds were purchased from Sigma-Aldrich and dissolved in room-temperature deionized water. Sucrose octaacetate was dissolved in warm water. Capsaicin was first dissolved in 100% ethanol and then diluted with water (final ethanol concentration, 0.5%). Tenofovir Alafenamide (TAF) and Praziquantel were synthesized by Nanjing Bilatchem Industrial Co. The TAF was first dissolved in DMSO to make a 1 M solution, which was then diluted with deionized water to the required concentrations. Praziquantel was sparingly soluble at the highest concentrations used here; it was dissolved in warm water. Ferroquine was obtained from Sanofi Pasteur Pharmaceutical Company; the powder was first dissolved in 50 mM HCl and then diluted in deionized water (final HCl concentration 1 mM).

The four concentrations (including deionized water) of a tastant were presented in a quasi-random order (a concentration could appear only once in a sequence of four tests). For each exposure, the gustometer shutter was open for 10 sec, during which licks of the drinking spout were counted. This was followed by 7.5 s with the shutter closed, during which a new taste solution was positioned, ready for the next presentation. Interposed between these test trials were 5-sec washout trials with water. Thus, a mouse received access to a taste solution for 10 sec followed by 7.5 sec with the shutter closed, then access to water for 5 sec followed by 7.5 sec with the shutter closed, followed by access to the next taste solution for 10 sec, and so on. We think the 5-sec washout trials with water prevent the mouse from quitting licking prematurely because it expects only bad-tasting solutions.

All sessions lasted 15 min but most mice stopped responding in the first 5 min. After a test session, each mouse received water for 1 h in its home cage and it was then deprived of water in preparation for the next session the following day.

Saccharin is considered hedonically positive by C57BL/6J mice and so it was tested using procedures different from those used with the other compounds. Following methods described in detail (31), prior to a session with saccharin, each mouse received free access to food and water for 24 h. It then received 1 g of food and 2 ml of water, and the session began 24 h later. In test sessions with saccharin there were no washout trials. After these sweet sessions, the mice had a recovery day with free access to food and water for 24 h.

### Statistical Analyses

For the series of experiments designed to find optimal test parameters, the dependent variable was the number of licks made during each 10-sec test. Analyses were based on the mean number of licks made by each mouse to deionized water and to 1 mM QHCl during each 10-sec test that the mouse responded, tests on which the mouse did not respond were excluded. The mean values for individual mice were then used in within-subject two-way analyses of variance with factors of condition [e.g., dose of AF-353 (with four levels)] and taste solution (with two levels: either deionized water or 1 mM QHCl). For the experiments involving IP injections and different vehicles, the ANOVAs involved three factors: route (oral or IP)×AF-353 (vehicle or AF-353)×fluid licked (deionized water or 1 mM QHCl) or vehicle (DMSO or alcohol+Tween)×AF-353 (vehicle or AF-353)×fluid licked (deionized water or 1 mM QHCl).

For the series of experiments designed to investigate AF-353's effect on licking for various taste compounds, the mean number of licks made by each mouse in response to each concentration was obtained by averaging together the results from identical 10-sec tests. To avoid the problem of expressing lick rates for deionized water (i.e., “0”) on a log scale, we converted all values to difference-from-water scores (i.e., licks for taste solution—licks for deionized water). These values for individual mice were then used in within-subject mixed-design analyses of variance with factors of treatment (vehicle or 3 mM AF-353) and concentration (with three levels). Lick rates to water are provided in Table 1A and Table 1B. Table 1A and Table 1B shows results of two-way analyses of variance and licks to water of mice swabbed orally with vehicle or AF-353 and then given three concentrations of a tastant to lick. Mice that did not lick during any presentation of a particular concentration of a taste compound were excluded from statistical analyses.

TABLE 1A

| | AF-353 | | | Concentration | | |
|---|---|---|---|---|---|---|
| Tastant | df | F | p | df | F | p |
| Saccharin | 1.10 | 10.29 | 0.0094 | 2.20 | 10.80 | 0.0007 |
| QHCl | 1.15 | 9.33 | 0.0080 | 2.30 | 24.88 | 0.0000 |
| NaCl | 1.10 | 2.53 | 0.1427 | 2.20 | 36.84 | 0.0000 |
| Citric acid | 1.13 | 6.45 | 0.0246 | 2.26 | 108.84 | 0.0000 |
| Capsaicin | 1.15 | 1.44 | 0.2485 | 2.30 | 24.46 | 0.0000 |
| Urea | 1.14 | 4.85 | 0.0450 | 2.28 | 5.79 | 0.0078 |
| Denatonium | 1.15 | 44.35 | 0.0000 | 2.30 | 39.91 | 0.0000 |
| SOA | 1.12 | 6.75 | 0.0233 | 2.24 | 6.89 | 0.0043 |
| Tenofovir | 1.15 | 17.28 | 0.0008 | 2.30 | 45.17 | 0.0000 |
| Praziquantel | 1.14 | 9.76 | 0.0075 | 2.28 | 10.61 | 0.0004 |
| Ferroquine | 1.14 | 18.13 | 0.0008 | 2.28 | 13.27 | 0.0000 |

* Saccharin = sodium saccharin; QHCl = quinine hydrochloride; Denatonium = denatonium benzoate; SOA = sucrose octaacetate; Tenofovir = Tenofovir Alafenamide. Licks to water = mean ± SE licks made in 10-sec tests. n = group size.

TABLE 1B

| | AF-353 × Concentration | | | | Licks to water | |
|---|---|---|---|---|---|---|
| Tastant | df | F | p | n | Vehicle | AF-353 |
| Saccharin | 2.20 | 6.90 | 0.0053 | 11 | 9 ± 2 | 14 ± 2 |
| QHCl | 2.30 | 1.99 | 0.1543 | 16 | 55 ± 7 | 56 ± 4 |
| NaCl | 2.20 | 4.11 | 0.0319 | 11 | 54 ± 6 | 57 ± 5 |
| Citric acid | 2.26 | 4.13 | 0.0277 | 14 | 52 ± 5 | 44 ± 4 |
| Capsaicin | 2.30 | 0.18 | 0.8383 | 16 | 48 ± 7 | 50 ± 9 |
| Urea | 2.28 | 7.95 | 0.0018 | 15 | 45 ± 6 | 43 ± 5 |
| Denatonium | 2.30 | 7.37 | 0.0025 | 13 | 65 ± 3 | 53 ± 3 |
| SOA | 2.24 | 5.07 | 0.0145 | 13 | 51 ± 4 | 47 = 4 |
| Tenofovir | 2.30 | 36.48 | 0.0000 | 16 | 57 ± 4 | 56 ± 2 |
| Praziquantel | 2.28 | 7.37 | 0.0025 | 15 | 50 ± 3 | 52 ± 3 |
| Ferroquine | 2.28 | 1.05 | 0.3632 | 15 | 45 ± 5 | 47 ± 3 |

* Saccharin = sodium saccharin; QHCl = quinine hydrochloride; Denatonium = denatonium benzoate; SOA = sucrose octaacetate; Tenofovir = Tenofovir Alafenamide. Licks to water = mean ± SE licks made in 10-sec tests. n = group size.

Post hoc least-significant difference tests were used to assess differences between means in licking rates (STATISTICA13.5, Stat Soft Inc.). All analyses were conducted using a criterion for significance of p<0.05.

### EXAMPLE 4—Investigation of P2X2/P2X3 Antagonists as Bitter Blockers—RESULTS Determining the Optimal Parameters to Observe an Effect of AF-353 on Bitter Taste Duration Swabbed

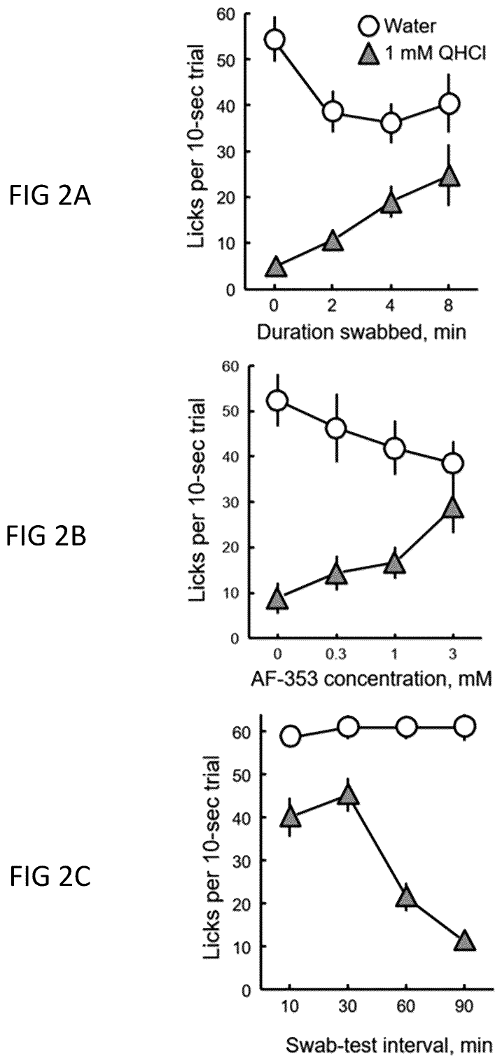

FIGS. 2A to 2C show results of the experiments of parameters for delivering AF-353 to the oral cavity. FIG. 2A shows influence on licking for deionized water and 1 mM quinine hydrochloride (QHCl) of the varied duration the oral cavity was swabbed (0-8 minutes). FIG. 2B shows influence on licking for deionized water and 1 mM quinine hydrochloride (QHCl) of the varied concentration of AF-353 swabbed orally for 4 min. FIG. 2C shows influence on licking for deionized water and 1 mM quinine hydrochloride (QHCl) of the interval between the end of swabbing the oral cavity with 3 mM AF-353 for 4 min and the start of gustometer testing. Values are means±SEs.

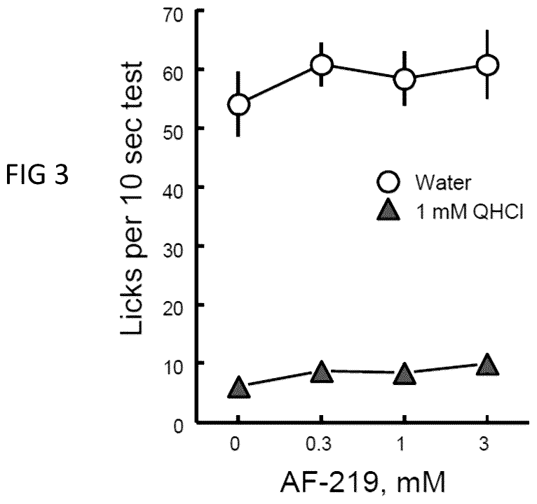

FIG. 3 shows influence on licking for deionized water and 1 mM quinine hydrochloride (QHCl) of the varied concentration of AF-219 swabbed orally.

Swabbing the oral cavity with AF-353 decreased licking for deionized water and increased licking for 1 mM QHCl [Swab duration×fluid licked interaction, F(3,27)=7.66, p=0.0007; FIG. 2A]. Mice that were not swabbed with 1 mM AF-353 (i.e., were treated for 0 mM QHCl (5±1 licks/10 sec; FIG. 2A). Relative to these control values, swabbing the oral cavity for 2, 4, or 8 min reduced lick rates for deionized water significantly, with the three durations being equally effective. In contrast, lick rates for 1 mM QHCl were increased monotonically, with 2 min of swabbing increasing 1 mM QHCl lick rates significantly above those without swabbing, 4 min of swabbing increasing 1 mM QHCl lick rates significantly above those following 2 min of swabbing, and 8 min of swabbing increasing 1 mM QHCl lick rates significantly above those following 4 min of swabbing. Based on these results, we chose to swab the oral cavity for 4 min in all subsequent experiments.

### AF-353 Concentration Swabbed

AF-353 applied to the oral cavity produced a concentration-related increase in lick rate for 1 mM QHCl and no significant effects on lick rates for deionized water [Concentration×fluid interaction, F(3,27)=3.34, p=0.0338; FIG. 2B]. Mice licked for 1 mM QHCl significantly more after 0.3 mM AF-353 than vehicle was applied to the oral cavity, significantly more after 1 mM than 0.3 mM AF-353, and significantly more after 3 mM than 1 mM AF-353. After treatment with the 3 mM concentration, lick rates for 1 mM QHCl and for deionized water were statistically indistinguishable. Based on this, we chose to swab the oral cavity with 3 mM AF-353 in subsequent experiments.

### Swab-Test Interval

The interval interposed between swabbing the oral cavity with AF-353 and the start of testing in the gustometer had no effect on lick rates for water but significantly influenced lick rates for 1 mM QHCl [Interval×fluid interaction, F(3,33)=31.4, p<0.0001; FIG. 2C]. There was no difference in lick rates for 1 mM QHCl between a 10-min or a 30-min swab-test interval; interposing a 60-min interval significantly reduced QHCl licking significantly relative to 1 mM QHCl licking after the 10- and 30-min intervals, and interposing a 90-min interval reduced 1 mM QHCl licking significantly relative to licking after a 60-min interval. Lick rates after a 90-min swab-test interval were similar to those observed in mice not treated with AF-353 in the previous two experiments (FIGS. 2A and 2B), suggesting that the effectiveness of AF-353 had completely dissipated at 90 min after oral treatment. Based on these results, we chose to interpose a 10-min interval between the end of swabbing the oral cavity and the beginning of behavioral testing.

### Oral Swab Versus Intraperitoneal Injection of AF-353

FIGS. 4A to 4B show effect of route of administration and vehicle for AF-353 on licking for deionized water and 1 mM quinine hydrochloride (QHCl). Values are means±SEs. FIG. 4A shows results of the experiment, plotted as licks per 10-sec trial, in which mice were administered 41 μmol/mouse AF-353 by oral swabbing. FIG. 4B shows results of the experiment, plotted as licks per 10-sec trial, in which mice were administered 41 μmol/mouse AF-353 by intraperitoneal injection. FIG. 4C shows results of the experiment, plotted as licks per 10-sec trial, in which mice were administered orally with AF-353 dissolved with DMSO. FIG. 4D shows results of the experiment, plotted as licks per 10-sec trial, in which mice were administered orally with AF-353 dissolved in alcohol with tween.

Lick rates depended on the interaction of the route of administration, what was administered, and what was available to lick [Route×AF-353× fluid interaction, F(1,15)=15.0, p=0.0015; FIGS. 4A and 4B]. Mice licked significantly more for 1 mM QHCl after AF-353 administration by either route than after DMSO vehicle; however, they licked for 1 mM QHCl significantly more after AF-353 administered orally than the same dose of AF-353 administered intraperitoneally. In this experiment, oral swabbing with AF-353 reduced licking relative to oral swabbing with DMSO vehicle (FIG. 4A).

### DMSO Versus Alcohol+Tween Vehicle

The composition of the vehicle for AF-353 did not influence licking rates [Vehicle×AF-353× Fluid interaction, F(1,9)=0.40, p=0.54 (NS; FIGS. 4C and 4D)]. The mice licked water near maximally irrespective of the vehicle or whether they received vehicle alone or vehicle plus AF-353. When given 1 mM QHCl to lick, they barely licked at all after being swabbed with either vehicle; they licked significantly more after oral AF-353 [AF-353× Fluid interaction, F(1, 9)=265.3, p<0.0001; FIGS. 4C and 4D].

### Determining the Effect of AF-353 on Exemplars of Basic Tastes, Representative Bitter Compounds, and Medicines

FIGS. 5A to 5E show results of licking for exemplars of four basic tastes and capsaicin by mice that have been orally swabbed with DMSO vehicle or 3 mM AF-353. Symbols show means±SEs; X-axis values are in mM. *p<0.05 relative to licks in vehicle-treated condition. FIG. 5A shows results of licking for sweet taste (saccharin). FIG. 5B shows results of licking for bitter taste (quinine hydrochloride (QHCL)). FIG. 5C shows results of licking for salty taste (NaCl). FIG. 5D shows results of licking for acidic (citric acid). FIG. 5E shows results of licking for capsaicin.

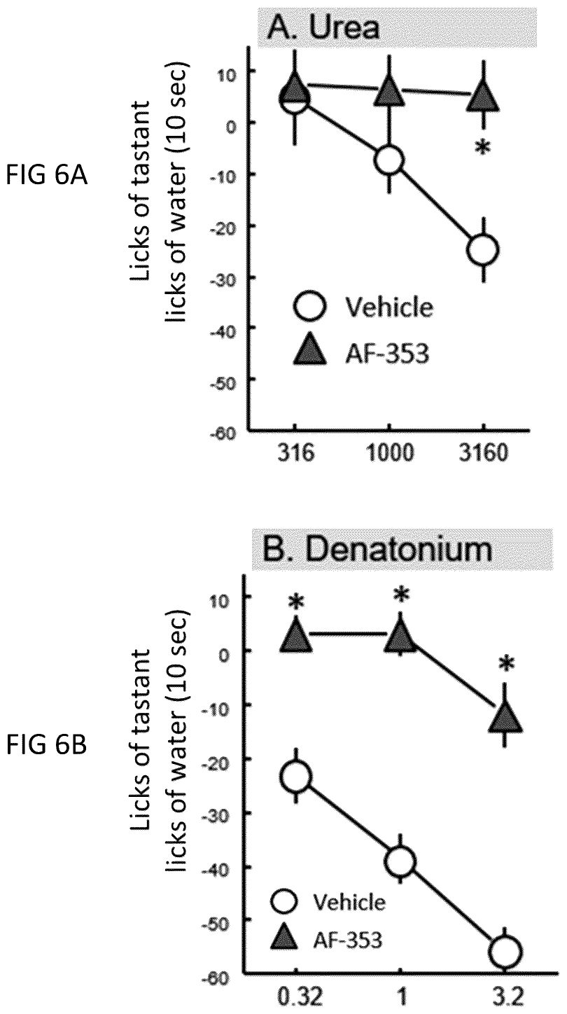

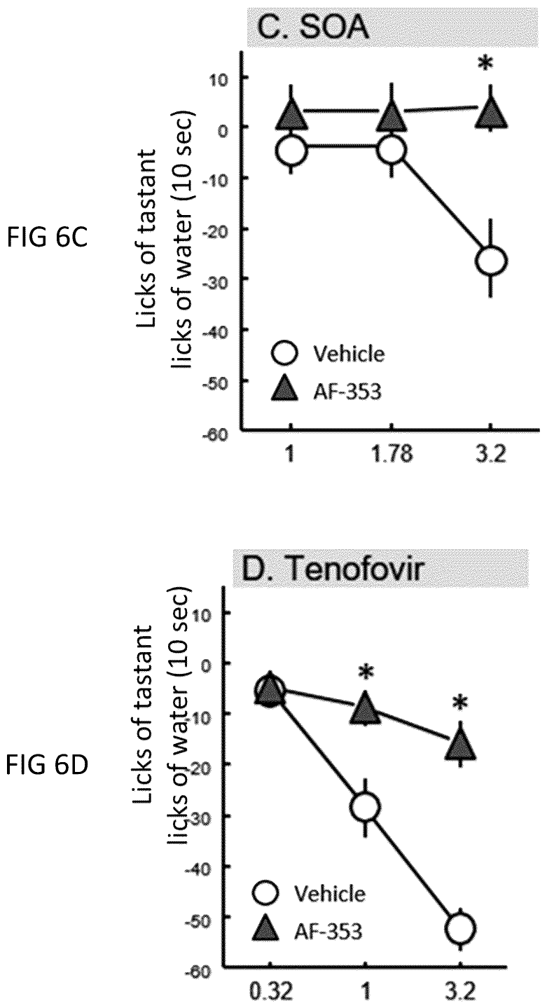

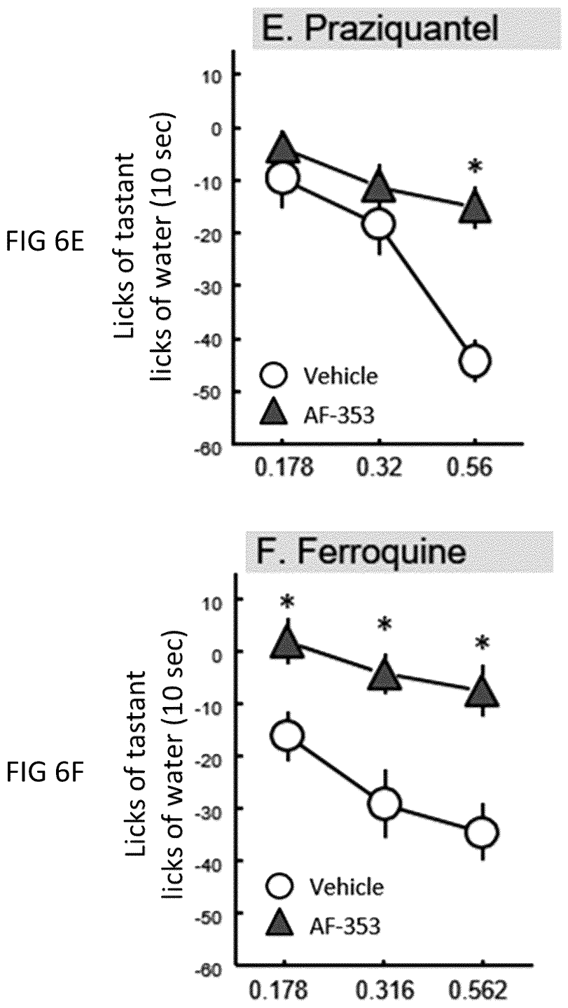

FIGS. 6A to 6F show licking for three bitter compounds and three medicines by mice that have been orally swabbed wth DMSO vehicle or 3 mM AF-353. Symbols show means±SEs; X-axis values are in mM.*p<0.05 relative to licks in vehicle-treated condition. FIG. 6A shows results of licking for urea. FIG. 6B shows results of licking for denatonium. FIG. 6C shows results of licking for sucrose octaacetate (SOA). FIG. 6D shows results of licking for Tenofovir. FIG. 6E shows results of licking for Praziquantel. FIG. 6F shows results of licking for Ferroquine.

Mice licked in a concentration-dependent manner for all of the tastants tested (Table 1A and Table 1B, FIGS. 5A-5E and 6A to 6F). Licking rates were decreased more by high than low concentrations of the exemplar bitter compounds (QHCl, urea, denatonium benzoate, sucrose octaacetate), medicines (Tenofovir Alafenamide, Praziquantel, Ferroquine), the sour tastant, citric acid, the salty tastant, NaCl, and the trigeminal agonist, capsaicin. Licking rates were increased more by high than low concentrations of saccharin. Relative to mice tested after being swabbed with DMSO vehicle, the same mice tested after swabbing their oral cavity with 3 mM AF-353 for 4 min licked more of all the hedonically negative tastants except NaCl and capsaicin, and licked less of the hedonically positive tastant, saccharin; Table 1A and Table 1B; FIG. 5A to 5E). In most cases (all except QHCl, Ferroquine, and capsaicin) the magnitude of the effect of AF-353 depended on the concentration of tastant being licked (Table 1A and Table 1B; FIGS. 5A-5E and 6A to 6F Capsaicin was the only tastant that AF-353 had neither a main effect of AF-353 nor an AF-353× concentration interaction (Table 1A and Table 1B; FIGS. 5A-5E).

### EXAMPLE 5—Investigation of Building Stable Cell Lines Expressing P2X2/3

In this study, we built stable cell lines expressing P2X2/3 in HEK293 cells via transfection with either P2X2-Neo or P2X3-Zeo constructs, following which selection was performed using Zeocin, G418. Picked clones were grown out from single cells (cultured continuously in the presence of Zeocin and G418) using cloning rings.

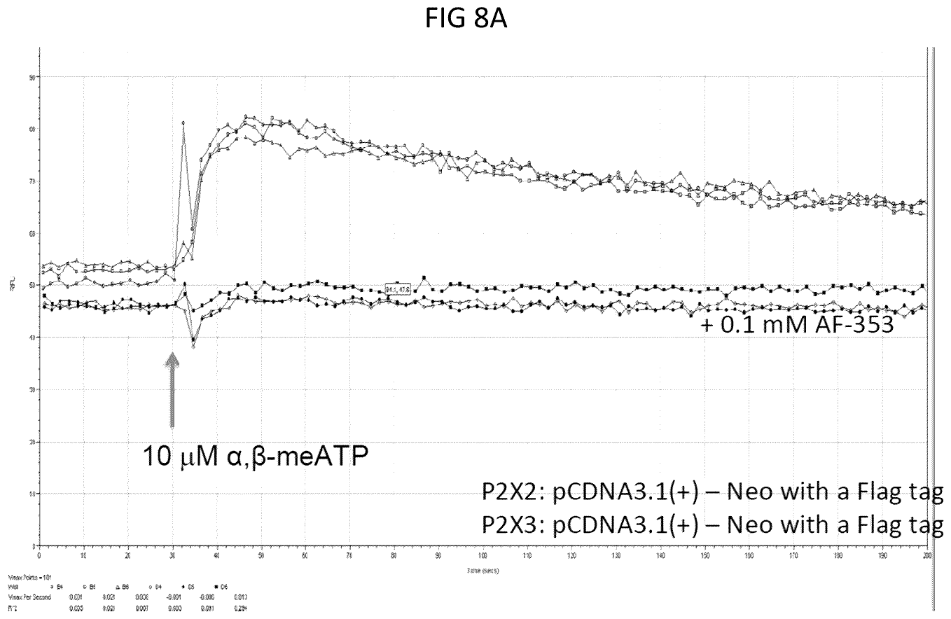

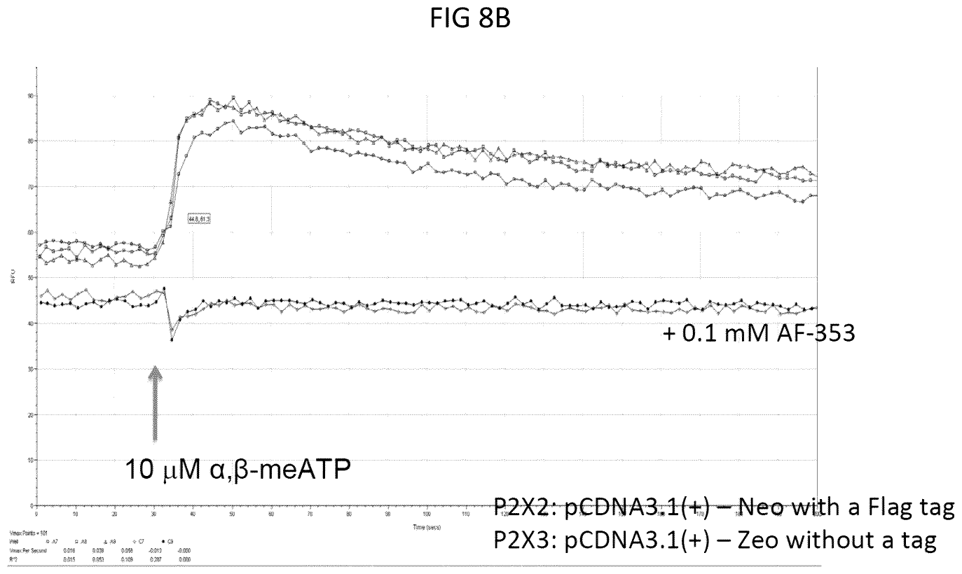

The resulted stable cell lines showed response to α,β-meATP and AF-353 in a dose-dependent manner (FIGS. 8A and 8B). The resulted stable cell lines are used for characterizing putative taste-blockers.

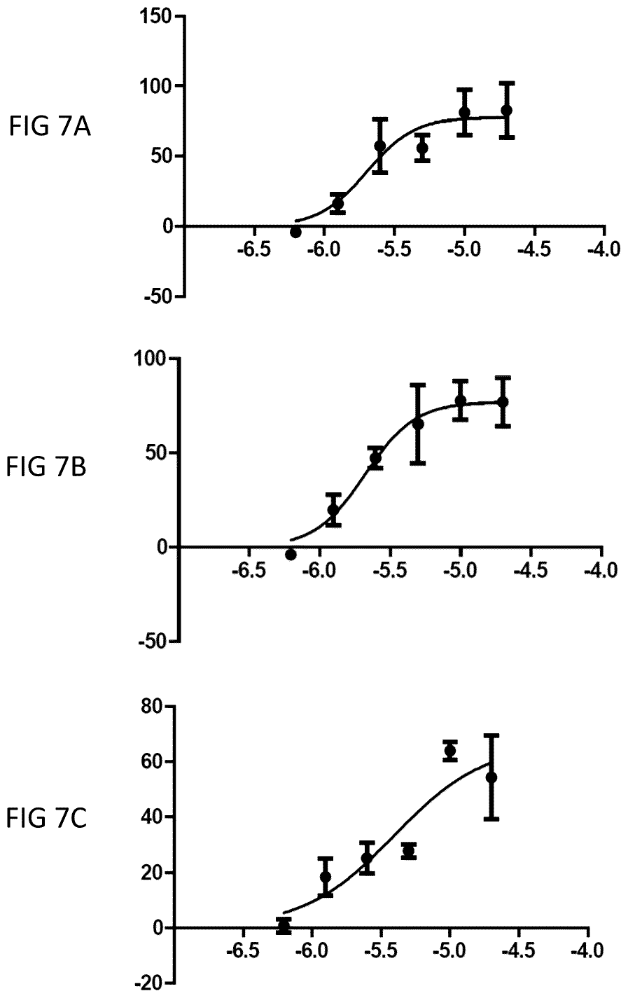

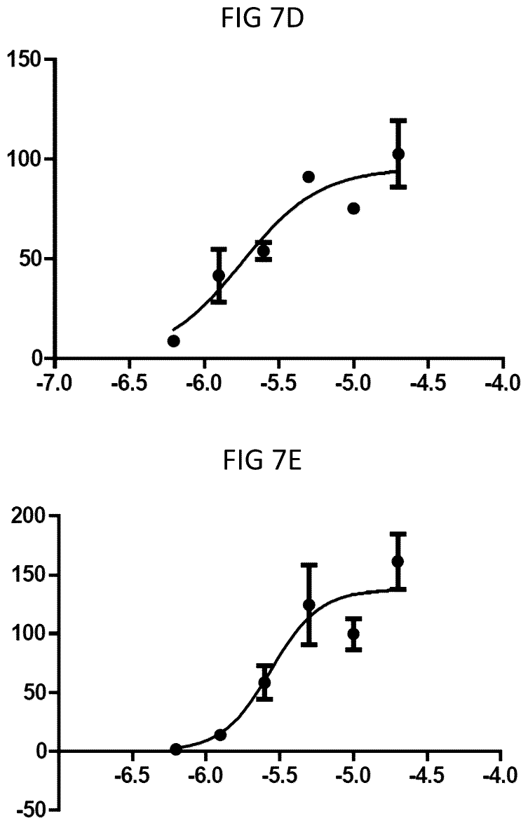

FIGS. 7A to 7E show expended various clones of P2X2/3 stable lines responding to α,β-meATP at a dose-dependent manner. FIG. 7A shows IC50 plot for P2X2/3 stable Line 2 (2.010e-006 M). FIG. 7B shows IC50 plot for P2X2/3 stable Line 6 (2.102e-006 M). FIG. 7C shows IC50 plot for P2X2/3 stable Line 9 (3.931e-006 M).FIG. 7D shows IC50 plot for P2X2/3 stable Line 12 (1.776e-006 M). FIG. 7E shows IC50 plot for P2X2/3 stable Line 24 (2.755e-006 M).

FIG. 8A shows exemplary screening assay results using one of the various clones of P2X2/3 stable lines responding to α,β-meATP in a dose-dependent manner in the presence or absence of AF-353. FIG. 8B shows another exemplary result of the screening assay for P2X2/P2X3 response to α,β-meATP in the presence or absence of AF-353 using P2X2/3 stable cell line.

### REFERENCES

Abdulgawi, R., et al., P2X3 receptor antagonist (AF-219) in refractory chronic cough: a randomized, double-blind, placebo-controlled phase 2 study, Lancet, 2015, 385: 1198-1205, ePub Nov. 25, 2014.
Ahmad, R., and Dalziel, J. E., G Protein-Coupled Receptors in Taste Physiology and Pharmacology, Frontiers in Pharmacology, 2020, 11: 587664, Pub Nov. 30, 2020.
Eddy, M. C., et al., Double P2X2/P2X3 Purinergic Receptor Knockout Mice Do Not Taste NaCl or the Artificial Sweetener SC45647, Chem Senses. 2009 November; 34(9): 789-797.
Ford, A. P., In pursuit of P2X3 antagonists: novel therapeutics for chronic pain and afferent sensitization, Purinergic Signaling, 2012, 8(1): S3-S26, epub Nov. 18, 2011.
Ford, A. P., et al., The discovery and development of gefapixant, Autonomic Neuroscience: Basic and Clinical, 235 (2021):102359.
Gever, J. R., et al., AF-353, a novel, potent and orally bioavailable P2X3/P2X2/3 receptor antagonist, Br J Pharmacol 2010 Jul; 160(6): 1387-1398.
Larson, E. D., et al., Function, Innervation, and Neurotransmitter Signaling in Mice Lacking Type-II Taste Cells, eNeuro. 2020 Jan-Feb; 7(1): ENEURO.0339-19.2020. Pre-published online 2020 Jan 23. Published online 2020 Jan 30.
Marucci, G., et al., Update on novel purinergic P2X3 and P2X2/X3 receptor antagonist and their potential therapeutic applications, Expert Opinion on Therapeutic Patents, 2019, 29(12): 943-963, ePub Nov. 14, 2019.
Schweiebert, E., et al., Inhibition of Bitter Taste from Oral Tenofavir Alafenamide, Molecular Pharmacology, 2021, 99(5):319-327.
Sclafani, A., and Ackoff, K., Fructose appetition in “taste-blind” P2X2/P2X3 double knockout mice, bioRxiv, 2021.07.22.453428.
Stokes, L., et al., To Inhibit or Enhance? Is there a Benefit to Positive Allosteric Modulation of P2X Receptors?, Frontiers in Parmacology, 2020, 11:627, Pub May 12, 2020.
Vandenbeuch, A., et al., Postsynaptic P2X3-containing receptors in gustatory nerve fibres mediate responses to all taste qualities in mice, J Physiol. 2015 Mar. 1; 593(Pt 5): 1113-1125. Published online 2015 Jan 20.
Abdulgawi, R.; Dockry, R.; Holt, K.; Layton, G.; McCarthy, B. G.; Ford, A. P.; Smith, J. A. P2X3 receptor antagonist (AF-219) in refractory chronic cough: a randomised, double-blind, placebo-controlled phase 2 study. Lancet 385:1198-1205; 2015.
Bo, X.; Alavi, A.; Xiang, Z.; Oglesby, I.; Ford, A.; Burnstock, G. Localization of ATP-gated P2X2 and P2X3 receptor immunoreactive nerves in rat taste buds. Neuroreport 10:1107-1111; 1999.
Cockayne, D. A.; Dunn, P. M.; Zhong, Y.; Rong, W.; Hamilton, S. G.; Knight, G. E.; Ruan, H.
Z.; Ma, B.; Yip, P.; Nunn, P.; McMahon, S. B.; Burnstock, G.; Ford, A. P. P2X2 knockout mice and P2X2/P2X3 double knockout mice reveal a role for the P2X2 receptor subunit in mediating multiple sensory effects of ATP. J Physiol 567:621-639; 2005.
Cui, W. W.; Wang, S. Y.; Zhang, Y. Q.; Wang, Y.; Fan, Y. Z.; Guo, C. R.; Li, X. H.; Lei, Y. T.; Wang, W. H.; Yang, X. N.; Hattori, M.; Li, C. Z.; Wang, J.; Yu, Y. P2X3-selective mechanism of Gefapixant, a drug candidate for the treatment of refractory chronic cough. Comput Struct Biotechnol J 20:1642-1653; 2022.
Eddy, M. C.; Eschle, B. K.; Barrows, J.; Hallock, R. M.; Finger, T. E.; Delay, E. R. Double P2X2/P2X3 purinergic receptor knockout mice do not taste NaCl or the artificial sweetener SC45647. Chem. Senses 34:789-797; 2009.
Ferguson, A. C.; Sutton, B. W.; Boone, T. B.; Ford, A. P.; Munoz, A. Inhibition of urothelial P2X3 receptors prevents desensitization of purinergic detrusor contractions in the rat bladder. BJU Int 116:293-301; 2015.
Finger, T. E.; Danilova, V.; Barrows, J.; Bartel, D. L.; Vigers, A. J.; Stone, L.; Hellekant, G.; Kinnamon, S. C. ATP signaling is crucial for communication from taste buds to gustatory nerves. Science 310:1495-1499; 2005.
Freund, J. R.; Lee, R. J. Taste receptors in the upper airway. World J Otorhinolaryngol Head Neck Surg 4:67-76; 2018.
Garceau, D.; Chauret, N. BLU-5937: A selective P2X3 antagonist with potent anti-tussive effect and no taste alteration. Pulm Pharmacol Ther 56:56-62; 2019.
Gever, J. R.; Soto, R.; Henningsen, R. A.; Martin, R. S.; Hackos, D. H.; Panicker, S.; Rubas, W.; Oglesby, I. B.; Dillon, M. P.; Milla, M. E.; Burnstock, G.; Ford, A. P. AF-353, a novel, potent and orally bioavailable P2X3/P2X2/3 receptor antagonist. Br. J. Pharmac 160:1387-1398; 2010.
Glendinning, J. I.; Gresack, J.; Spector, A. C. A high-throughput screening procedure for identifying mice with aberrant taste and oromotor function. Chem. Senses 27:461-474; 2002.
Hallock, R. M., et al., Residual chemosensory capabilities in double P2X2/P2X3 purinergic receptor null mice: intraoral or postingestive detection? Chem. Senses 34:799-808; 2009.
Ishida, Y.; Ugawa, S.; Ueda, T.; Yamada, T.; Shibata, Y.; Hondoh, A.; Inoue, K.; Yu, Y.; Shimada, S. P2X(2)—and P2X(3)-positive fibers in fungiform papillae originate from the chorda tympani but not the trigeminal nerve in rats and mice. J. Comp. Neurol. 514:131-144; 2009.
Kaan, T. K.; Yip, P. K.; Grist, J.; Cefalu, J. S.; Nunn, P. A.; Ford, A. P.; Zhong, Y.; McMahon, S. B. Endogenous purinergic control of bladder activity via presynaptic P2X3 and P2X2/3 receptors in the spinal cord. J Neurosci 30:4503-4507; 2010.
Kaan, T. K.; Yip, P. K.; Patel, S.; Davies, M.; Marchand, F.; Cockayne, D. A.; Nunn, P. A.; Dickenson, A. H.; Ford, A. P.; Zhong, Y.; Malcangio, M.; McMahon, S. B. Systemic blockade of P2X3 and P2X2/3 receptors attenuates bone cancer pain behaviour in rats. Brain 133:2549-2564; 2010.
Klein, S.; Gashaw, I.; Baumann, S.; Chang, X.; Hummel, T.; Thuss, U.; Friedrich, C. First-in-human study of eliapixant (BAY 1817080), a highly selective P2X3 receptor antagonist: tolerability, safety and pharmacokinetics. Br. J. Cli. Pharmac.; 2022.
Larson, E. D., et al., Function, innervation, and neurotransmitter signaling in mice lacking Type-II taste cells. eNeuro 7; 2020.
Ma, Z.; Taruno, A.; Ohmoto, M.; Jyotaki, M.; Lim, J. C.; Miyazaki, H.; Niisato, N.; Marunaka, Y.; Lee, R. J.; Hoff, H.; Payne, R.; Demuro, A.; Parker, I.; Mitchell, C. H.; Henao-Mejia, J.; Tanis, J. E.; Matsumoto, I.; Tordoff, M. G.; Foskett, J. K. CALHM3 is essential for rapid ion channel-mediated purinergic neurotransmission of GPCR-mediated tastes. Neuron 98:547-561 e510; 2018.
Med Associates inc. Products: Gustometry. https://www.med-associates.com/product/davis-rig-for-mouse-16-bottle/. 2022.
Morice, A.; Smith, J. A.; McGarvey, L.; Birring, S. S.; Parker, S. M.; Turner, A.; Hummel, T.; Gashaw, I.; Fels, L.; Klein, S.; Francke, K.; Friedrich, C. Eliapixant (BAY 1817080), a P2X3 receptor antagonist, in refractory chronic cough: a randomised, placebo-controlled, crossover phase 2a study. Eur Respir J 58; 2021.
Munoz, A.; Somogyi, G. T.; Boone, T. B.; Ford, A. P.; Smith, C. P. Modulation of bladder afferent signals in normal and spinal cord-injured rats by purinergic P2X3 and P2X2/3 receptors. BJU Int 110:E409-414; 2012.
Ohkuri, T., et al., Residual chemoresponsiveness to acids in the superior laryngeal nerve in “taste-blind” (P2X2/P2X3 double-KO) mice. Chem. Senses 20:523-532; 2012.
Riera, C. E., et al., The capsaicin receptor participates in artificial sweetener aversion. Biochem. Biophys. Res. Comm. 376:653-657; 2008.
Salazar, B. H.; Hoffman, K. A.; Zhang, C.; Kavanagh, A.; Zhang, Y.; Boone, T. B.; Munoz, A. Electrical activity of the bladder Is attenuated by intravesical inhibition of P2X2/3 receptors during micturition in female rats. Int Neurourol J 21:259-269; 2017.
Salazar, B. H.; Hoffman, K. A.; Zhang, C.; Zhang, Y.; Cruz, Y.; Boone, T. B.; Munoz, A. Modulatory effects of intravesical P2X2/3 purinergic receptor inhibition on lower urinary tract electromyographic properties and voiding function of female rats with moderate or severe spinal cord injury. BJU Int 123:538-547; 2019.
Sclafani, A.; Ackroff, K. Maltodextrin and fat preference deficits in “taste-blind” P2X2/P2X3 knockout mice. Chem. Senses; 2014.
Sclafani, A.; Marambaud, P.; Ackroff, K. Sucrose-conditioned flavor preferences in sweet ageusic T1r3 and Calhml knockout mice. Physiol Behav 126:25-29; 2014.
Sheridan, C. Merck stakes out ‘irritable’ neuron territory with $1.25 billion. Nature biotechnology 34:900; 2016.
Smith, J. C. The history of the “Davis Rig”. Appetite 36:93-98; 2001.
Stratford, J. M.; Finger, T. E. Central representation of postingestive chemosensory cues in mice that lack the ability to taste. J Neurosci 31:9101-9110; 2011.
Taruno, A., et al., CALHM1 ion channel mediates purinergic neurotransmission of sweet, bitter and umami tastes. Nature 495:223-226; 2013.
Tordoff, M. G. Metabolic basis of learned food preferences. In: Friedman, M. I.; Kare, M. R.;
Tordoff, M. G., eds. Chemical senses: appetite and nutrition. Vol. 4. New York: Marcel Dekker; 1991:239-260.
Tordoff, M. G., et al., Normal taste acceptance and preference of PANX1 knockout mice. Chem. Senses 2015 May 18. pii: bjv025; 2015.
Tordoff, M. G.; Aleman, T. R.; McCaughey, S. A. Heightened avidity for trisodium pyrophosphate in mice lacking Taslr3. Chem. Senses 40:53-59; 2014.
Tordoff, M. G.; Bachmanov, A. A. Monell mouse taste phenotyping project. Monell Chemical Senses Center. www.monell.org/MMTPP. 2001.
Tordoff, M. G.; Ellis, H. T. Taste dysfunction in BTBR mice due to a mutation of Itpr3, the inositol triphosphate receptor 3 gene. Physiol. Genomics 45:834-855; 2013.
Tordoff, M. G.; Ellis, H. T.; Aleman, T. R.; Downing, A.; Marambaud, P.; Foskett, J. K.; Dana, R. M.; McCaughey, S. A. Salty taste deficits in CALHM1 knockout mice. Chem. Senses 39:515-528; 2014.
Vandenbeuch, A., et al., Postsynaptic P2X3-containing receptors in gustatory nerve fibres mediate responses to all taste qualities in mice. J Physiol 593:1113-1125; 2015.
Weigand, L. A.; Ford, A. P.; Undem, B. J. A role for ATP in bronchoconstriction-induced activation of guinea pig vagal intrapulmonary C-fibres. J Physiol 590:4109-4120; 2012.

All documents cited in this specification are incorporated herein by reference. U.S. Provisional Patent Application No. 63/251,999, filed Oct. 4, 2021 is incorporated herein by reference in its entirety. While the invention has been described with reference to particular embodiments, it will be appreciated that modifications can be made without departing from the spirit of the invention. Such modifications are intended to fall within the scope of the appended claims.

1. A method of reducing bitter taste attributed to a bitter taste of an active pharmaceutical ingredient (API) in a medicament, nutrient, and/or a dietary supplement, said method comprising administration of the taste-masking composition, wherein said taste-masking composition comprises:
at least one compound that is a purinergic receptor inhibitor, wherein the at least one compound modulates an aversive taste of an API in a medicament, nutrient, and/or a dietary supplement which is to be administered to a subject in a need thereof; and
a pharmaceutically acceptable carrier,
wherein the at least one compound is in an amount sufficient to reduce and/or block the aversive taste of the API,
wherein the taste-masking composition is formulated for oral administration and is administered prior to the or with the administration of the medicament and/or dietary supplement to the subject in a need thereof, and
wherein said taste-masking composition is administered before the administration of the medicament, nutrient, and/or the dietary supplement to a subject in a need thereof.

2. The method according to claim 1, wherein the purinergic receptor inhibitor targets a purinergic receptor which comprises a homomeric or a heteromeric purinergic receptor.

3. The method according to claim 1, wherein the purinergic receptor inhibitor targets a purinergic receptor which is a P2X purinergic receptor.

4. The method according to claim 1, wherein the purinergic receptor inhibitor targets a purinergic receptor which is selected from P2X1, P2X2, P2X3, P2X4, P2X5, P2X7, P2X2/P2X3, P2X4/P2X6, and/or P2X1/P2X5.

5. The method according to claim 1, wherein the purinergic receptor inhibitor modulates an aversive taste of a medication, nutrient, and/or a dietary supplement, wherein the aversive taste is perceived by the human subject as sweet, salty, sour, savory, and/or bitter.

6. The method according to claim 1, wherein the purinergic receptor inhibitor is selected from arylamide derivatives, diaminpyrimidine derivatives, imidazo-pyrimidine derivatives, pyrazolo-pyrimidine derivatives, pyrazole derivatives, oxazole derivatives, pyridine derivatives, pyrimidine derivatives, and physiologically or pharmaceutically acceptable salts thereof.

7. The method according to claim 1, wherein the purinergic receptor inhibitor is 5-[5-iodo-4-methoxy-2-(1-methylethyl)phenoxy]-2,4-pyrimidinediamine (AF-353), optionally wherein the inhibitor is a derivative of 5-[5-iodo-4-methoxy-2-(1-methylethyl)phenoxy]-2,4-pyrimidinediamine.

8. The method according to claim 1, wherein the purinergic receptor inhibitor is administered to present orally in an amount sufficient to reduce and/or block the aversive taste of the API.

9. The method according to claim 1, wherein the purinergic receptor inhibitor is administered at a dose
(a) about 0.01 mg/mL to about 0.4 mg/mL;
(b) about 0.04 mg/mL to about 0.2 mg/mL; or
(c) about 0.1 mg/mL to about 0.15 mg/mL.

10. The method according to claim 1, wherein the medicament, nutrient, and/or a dietary supplement is selected from active antibiotic medicament, antibacterial medicament, anti-protozoal medicament, anti-amoebal, anti-fungal, anti-viral medicament, anti-helminth medicament, herbal medicament, and essential amino-acid medicament and/or supplement.

11. The method according to claim 1, wherein the taste-masking composition is formulated for topical oral administration in the form of a dissolving strip, a coating applied on a food product, a coating applied on an API, a chewable food product, a chewable or rapid dissolve tablet, a lollipop, a spray, a mist, or a liquid suspension.

12. The method according to claim 11, wherein the composition is formulated in a liquid suspension which is a neutral tasting oral rinse liquid suspension.

13. A composition which is a taste-masking and/or taste-blocking composition, said composition comprising:
at least one compound that is a purinergic receptor inhibitor, wherein the at least one compound modulates an aversive taste of an active pharmaceutical ingredient (API) in a medicament, nutrient, and/or a dietary supplement which is to be administered to a subject in a need thereof; and
a pharmaceutically acceptable carrier,
wherein the at least one compound is in an amount sufficient to reduce and/or block the aversive taste of the API, and
wherein the composition is formulated for oral administration and is administered prior to the or with the administration of the medicament and/or dietary supplement to the subject in a need thereof.

14. The composition according to claim 13, wherein the purinergic receptor inhibitor targets a purinergic receptor which comprises a homomeric or a heteromeric purinergic receptor.

15. The composition according to claim 13, wherein the purinergic receptor inhibitor targets a purinergic receptor which is a P2X purinergic receptor.

16. The composition according to claim 13, wherein the purinergic receptor inhibitor targets a purinergic receptor which is selected from P2X1, P2X2, P2X3, P2X4, P2X5, P2X7, P2X2/P2X3, P2X4/P2X6, and/or P2X1/P2X5.

17. The composition according to claim 13, wherein the purinergic receptor inhibitor modulates an aversive taste of a medication, nutrient, and/or a dietary supplement, wherein the aversive taste is perceived by the human subject as sweet, salty, sour, savory, and/or bitter.

18. The composition according to claim 13, wherein the purinergic receptor inhibitor is selected from arylamide derivatives, diaminpyrimidine derivatives, imidazo-pyrimidine derivatives, pyrazolo-pyrimidine derivatives, pyrazole derivatives, oxazole derivatives, pyridine derivatives, pyrimidine derivatives, and physiologically or pharmaceutically acceptable salts thereof.

19. The composition according to claim 13, wherein the purinergic receptor inhibitor is 5-[5-iodo-4-methoxy-2-(1-methylethyl)phenoxy]-2,4-pyrimidinediamine (AF-353), optionally wherein the inhibitor is a derivative of 5-[5-iodo-4-methoxy-2-(1-methylethyl)phenoxy]-2,4-pyrimidinediamine.

20. The composition according to claim 13, wherein the purinergic receptor inhibitor is present orally in an amount sufficient to reduce and/or block the aversive taste of the API.

21. The composition according to claim 13, wherein the purinergic receptor inhibitor is at a dose
(a) about 0.01 mg/mL to about 0.4 mg/mL;
(b) about 0.04 mg/mL to about 0.2 mg/mL; or
(c) about 0.1 mg/mL to about 0.15 mg/mL.

22. The composition according to claim 13, wherein the medicament, nutrient, and/or a dietary supplement is selected from active antibiotic medicament, antibacterial medicament, anti-protozoal medicament, anti-amoebal, anti-fungal, anti-viral medicament, anti-helminth medicament, herbal medicament, and essential amino-acid medicament and/or supplement.

23. The composition according to claim 13, wherein the composition is formulated for topical oral administration in the form of a dissolving strip, a coating applied on a food product, a coating applied on an API, a chewable food product, a chewable or rapid dissolve tablet, a lollipop, a spray, a mist, or a liquid suspension.

24. The composition according to claim 23, wherein the composition is formulated in a liquid suspension which is a neutral tasting oral rinse liquid suspension.

25. A method of blocking bitter taste attributed to a bitter taste of an API in a medicament, nutrient, and/or a dietary supplement, said method comprising administration of the taste-masking composition according to claim 13, wherein said taste-masking composition is administered before the administration of the medicament, nutrient, and/or the dietary supplement to a subject in a need thereof.

26. A method of modulating aversive taste attributed to an aversive taste of an API in a medicament, nutrient, and/or a dietary supplement, said method comprising administration of the taste-masking composition according to claim 13, wherein said taste-masking composition is administered before the administration of the medicament, nutrient, and/or the dietary supplement to a subject in a need thereof.

27. A method of facilitating taking a medicament, nutrient, and/or a dietary supplement which comprises an averse tasting API, said method comprising administration of the taste-masking composition according to claim 13, wherein said taste-masking composition is administered before the administration of the medicament, nutrient, and/or the dietary supplement to a subject in a need thereof.

28. A method of increasing patient compliance of taking a medicament, nutrient, and/or a dietary supplement which comprises an averse tasting API, said method comprising administration of the taste-masking composition according to claim 13, wherein said taste-masking composition is administered before the administration of the medicament, nutrient, and/or the dietary supplement to a subject in a need thereof.

29. The method according to claim 1, wherein the taste-masking composition is administered at least about 1 hour, at least about 30 minutes, at least about 10 minutes, at least about 5 minutes, or at least about 1 minutes prior to the administration of the medicament, nutrient, and/or the dietary supplement.

30. The method according to claim 1, wherein the taste-masking composition is administered at the same time with the administration of the medicament, nutrient, and/or a dietary supplement.

31. A regimen for taking a medicament, nutrient, and/or a dietary supplement which comprises an averse tasting API, said regimen comprising administering the taste-masking composition according to claim 13 to a patient in a need thereof at least about 1 hour, at least about 30 minutes, at least about 15 minutes, at least about 10 minutes, or at least about 5 minutes prior to the administration of the medicament, nutrient, and/or the dietary supplement.

32. A regimen for taking a medicament, nutrient, and/or a dietary supplement which comprises an averse tasting API, said regimen comprising administering the taste-masking composition according to claim 13 with the medicament, nutrient, and/or the dietary supplement.

33. The regimen according to claim 31, wherein the taste masking composition is admixed with the medicament, nutrient, and/or the dietary supplement, in a ratio amount sufficient to modulate, block or reduce the aversive taste of the API in the medicine and/or the dietary supplement.

34. A dissolving strip formulated for topical oral administration for use in facilitating taking a medicament, nutrient, and/or a dietary supplement that comprises an averse tasting API, wherein the dissolving strip comprises the taste masking composition according to claim 13.

35. A dissolving strip formulated for topical oral administration for use in increasing patient compliance of taking a medicament, nutrient, and/or a dietary supplement that comprises an averse tasting API, wherein the dissolving strip comprises the taste masking composition according to claim 13.

36. A coating applied on a food product formulated for topical oral administration for use in facilitating taking a medicament, nutrient, and/or a dietary supplement that comprises an averse tasting API, wherein the coating comprises the taste masking composition according to claim 13.

37. A coating applied on a food product formulated for topical oral administration for use in increasing patient compliance of taking a medicament, nutrient, and/or a dietary supplement that comprises an averse tasting API, wherein the coating comprises the taste masking composition according to claim 13.

38. A chewable food product formulated for topical oral administration for use in facilitating taking a medicament, nutrient, and/or a dietary supplement that comprises an averse tasting API, wherein the chewable food product comprises the taste masking composition according to claim 13.

39. A chewable food product formulated for topical oral administration for use in increasing patient compliance of taking a medicament, nutrient, and/or a dietary supplement that comprises an averse tasting API, wherein the chewable food product comprises the taste masking composition according to claim.

40. A lollipop formulated for topical oral administration for use in facilitating taking a medicament, nutrient, and/or a dietary supplement that comprises an averse tasting API, wherein the lollipop comprises the taste masking composition according to claim 13.

41. A lollipop formulated for topical oral administration for use in increasing patient compliance of taking a medicament, nutrient, and/or a dietary supplement that comprises an averse tasting API, wherein the lollipop comprises the taste masking composition according to claim 3.

42. A liquid suspension formulated for topical oral administration for use in in facilitating taking a medicament, nutrient, and/or a dietary supplement that comprises an averse tasting API, wherein the liquid suspension comprises the composition according to claim 13.

43. A liquid suspension formulated for topical oral administration for use in increasing patient compliance of taking a medicament, nutrient, and/or dietary supplement that comprises an averse tasting API, wherein the liquid suspension comprises the composition according to any.

44. The liquid suspension according to claim 42, wherein the liquid formulation is a mouthwash.

45. The liquid suspension according to claim 42, wherein the liquid formulation is an oral topical spray or an oral topical mist.

46. The liquid suspension according to claim 45, wherein the oral topical spray or the oral topical mist coats the epithelium of the oral cavity.

47. A chewable or rapid dissolve tablet formulated for topical oral administration for use in in facilitating taking a medicament, nutrient, and/or a dietary supplement that comprises an averse tasting API, wherein the liquid suspension comprises the composition according to claim 13.

48. A chewable or rapid dissolve tablet formulated for topical oral administration for use in increasing patient compliance of taking a medicament, nutrient, and/or dietary supplement that comprises an averse tasting API, wherein the liquid suspension comprises the composition according to claim 13.

49. A coating applied on an API formulated for topical oral administration for use in in facilitating taking a medicament, nutrient, and/or a dietary supplement that comprises an averse tasting API, wherein the liquid suspension comprises the composition according to claim 13.

50. A coating applied on an API formulated for topical oral administration for use in increasing patient compliance of taking a medicament, nutrient, and/or dietary supplement that comprises an averse tasting API, wherein the liquid suspension comprises the composition according to claim 13.
